(19) United States
(12) Patent Application Publication
(10) Pub. No.: US 2024/0101585 A1
(43) Pub. Date: Mar. 28, 2024

(54) HETEROAROMATIC PHOSPHONIUM SALTS AND THEIR USE TREATING CANCER

(71) Applicant: FLORATEK PHARMA AG, Stansstad (CH)

(72) Inventor: Dan Florin Stoicescu, Aubonne (CH)

(21) Appl. No.: 18/274,318

(22) PCT Filed: Jan. 26, 2022
(86) PCT No.: PCT/EP2022/051805

§ 371 Date: Jul. 26, 2023

(30) Foreign Application Priority Data
Jan. 26, 2021 (GB) 2101043.4

(51) Int. Cl. C07F 9/655; A61P 35/00

(52) U.S. Cl. CPC C07F 9/65522; A61P 35/00

(57) ABSTRACT

The present invention relates to chromen-4-one derivatives comprising a phosphonium quaternary group, and to associated multi-salts, solvates, prodrugs and pharmaceutical compositions. The present invention also relates to the use of such compounds and compositions in the treatment and prevention of cancer.

### FIELD OF THE INVENTION

The present invention relates to flavonoid compounds, and to associated multi-salts, solvates, prodrugs and pharmaceutical compositions. The present invention also relates to the use of such compounds and compositions in the treatment and prevention of cancer.

### BACKGROUND

Targeting delayed or inhibited apoptosis is a major approach in cancer treatment and a highly active area of research. Apoptosis is a stringently organized process, regulated by a series of signal transduction cascades and cellular proteins. Two major pathways contributing to apoptosis: firstly, the extrinsic/death receptor induced pathway and secondly, the intrinsic pathway in which mitochondrial stress is involved [Rathore R., McCallum J. E., Varghese E., Maria A., Büsselberg D. Overcoming chemotherapy drug resistance by targeting inhibitors of apoptosis proteins (iaps) Apoptosis. 2017; 22:898-919]. Mitochondrial pathway of apoptosis is the most commonly deregulated type of cell death in cancer, and the understanding of mitochondrial apoptosis had advanced, so that novel therapies can be developed to specifically activate this process. [Lopez J., Tait S. W. G. Mitochondrial apoptosis: Killing cancer using the enemy within. Br. J. Cancer. 2015; 112:957-962]. In healthy cells, mitochondria execute a controlled regulation of multiple functions to maintain the cellular growth-death cycle. However, in the case of tumour cells, to meet the higher metabolic demand of rapidly proliferating cells, dysregulation of mitochondrial metabolism occurs. The difference between cancer cell mitochondria and normal cells includes several functional alterations, such as mutation of mtDNA, deficient respiration and ATP generation, mutation of mtDNA-encoded mitochondrial enzymes and structural differences, such as higher membrane potential of cancer cell mitochondria and higher basicity inside the mitochondrial lumen. The evasion of cell death or inhibition of mitochondria-mediated apoptosis is a hallmark for cancer. Mitochondria generate ROS, which is necessary for signalling under normal conditions. However, when apoptosis is inhibited in the case of cancer, ROS contributes to the neoplastic transformation. This altered mitochondrial metabolism of cancer cells compared with that of their normal counterparts is advantageous for the selective targeting of cancer mitochondria in therapeutics, which focuses on the cancer mitochondria specific features [Rin Jean S., Tulumello D. V., Wisnovsky S. P., Lei E. K., Pereira M. P., Kelley S. O. Molecular vehicles for mitochondrial chemical biology and drug delivery. ACS Chem. Biol. 2014; 9:323-333]. Anticancer drugs that selectively disrupt cancerous mitochondria could be achieved by designing molecules that act on the malignant mitochondria by, for instance, inhibiting glycolysis, depolarizing the membrane potential, and inhibiting the mitochondrial permeability transition pore [Dilip A., Cheng G., Joseph J., Kunnimalaiyaan S., Kalyanaraman B., Kunnimalaiyaan M., Gamblin T. C. Mitochondria-targeted antioxidant and glycolysis inhibition: Synergistic therapy in hepatocellular carcinoma. Anticancer Drugs. 2013; 24:881-888].

There is a need to provide compounds with improved pharmacological and/or physiological and/or physiochemical properties and/or those that provide a useful alternative to known compounds.

### SUMMARY OF THE INVENTION

The present invention addresses the limitations of the polyphenol class of compounds in maximizing their natural anti-cancer potential by providing a series of structurally novel compounds targeted to the mitochondrial membrane, thus enhancing the apoptotic pathway and potentially overcoming drug resistance by bypassing the cells mechanism of evading the apoptotic pathway. The compounds are effective through a multi-targeted approach using the lipophilic ion to rapidly penetrate and accumulate in the mitochondrial membrane and the polyphenolic moiety to exert anti-oxidant and antiproliferative effects. Additionally or alternatively, the discovered compound series optimizes the alkyl linker used to connect the lipophilic ion with the biologically active moiety.

The present invention is defined in the claims.

A first aspect of the invention provides a compound of formula (I):

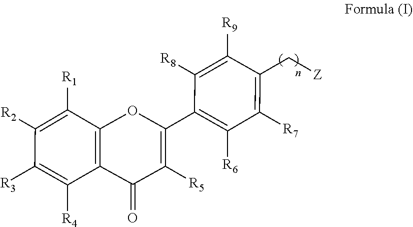

wherein:
Z is —[P(R¹¹)₃]X, wherein X is a counter anion;
R¹, R², and R⁴, independently, are selected from —OH, —O—C₁₋₄ alkyl, —OC(O)R₁₃, —OC(O)NHR¹³, —OC(O)N(R¹³)₂; or R¹ and R² together form —O—(C₁₋₃ alkylene)-O—;
R³, R⁵, R⁶, R⁷, R⁸, and R⁹, independently, are selected from H; halo; —CN; —NO₂; —R^{β}; —OH, —OR^{β}; —SH; —SR^{β}; —SOR^{β}; —SO₂H; —SO₂R^{β}; —SO₂NH₂; —SO₂NHR^{β}; —SO₂N(R^{β})₂; —NH₂; —NHR^{β}; —N(R^{β})₂; —CHO; —COR^{β}; —COOH; —COOR^{β}; —OCOR^{β}; and benzyl optionally substituted with 1-3 —R^{β};
each —R^{β} is independently selected from a C₁-C₆ alkyl, C₂-C₆ alkenyl, C₂-C₆ alkynyl or C₃-C₁₄ cyclic group, and wherein any —R^{β} may optionally be substituted with one or more C₁-C₄ alkyl, C₁-C₄ haloalkyl, C₃-C₇ cycloalkyl, —O(C₁-C₄ alkyl), —O(C₁-C₄ haloalkyl), —O(C₃-C₇ cycloalkyl), halo, —OH, —NH₂, —CN, —NO₂, —C≡CH, —CHO, —CON(CH₃)₂ or oxo (═O) groups;
each —R¹¹ is independently selected from H, C₁-C₆ alkyl, C₂-C₆ alkenyl, C₃-C₁₄ aryl group, or C₃-C₁₄ aliphatic cyclic group, and wherein any —R¹¹ may optionally be substituted with one or more C₁-C₄ alkyl, C₁-C₄ haloalkyl, C₃-C₇ cycloalkyl, —O(C₁-C₄ alkyl), —O(C₁-C₄ haloalkyl), —O(C₃-C₇ cycloalkyl), halo, —OH, —NH₂, —CN, —C≡CH or oxo (═O) groups
each —R¹³ is independently selected from a H, C₁-C₆ alkyl, C₂-C₆ alkenyl, C₂-C₆ alkynyl, C₃₋₁₄ cyclic group, halo, —NO₂, —CN, —OH, —NH₂, mercapto, formyl, carboxy, carbamoyl, C₁₋₆ alkoxy, C₁₋₆ alkylthio, —NH(C₁₋₆ alkyl), —N(C₁₋₆ alkyl)₂, C₁₋₆ alkylsulfinyl, C₁₋₆ alkylsulfonyl, or arylsulfonyl, wherein any —R¹³ may optionally be substituted with one or more —R¹⁴;
each R¹⁴ is independently selected from a C₁-C₆ alkyl, C₂-C₆ alkenyl, C₂-C₆ alkynyl, C₃₋₁₄ cyclic group, halo, —NO₂, —CN, —OH, —NH₂, mercapto, formyl, carboxy, carbamoyl, C₁₋₆ alkoxy, C₁₋₆ alkylthio, —NH(C₁₋₆ alkyl), —N(C₁₋₆ alkyl)₂, C₁₋₆ alkylsulfinyl, C₁₋₆ alkylsulfonyl, or arylsulfonyl, wherein any —R₁₄ may optionally be substituted with one or more —R₁₅;
each —R¹⁵ is independently selected from halogen, nitro, cyano, hydroxy, trifluoromethoxy, trifluoromethyl, amino, formyl, carboxy, carbamoyl, mercapto, sulfamoyl, methyl, ethyl, methoxy, ethoxy, acetyl, acetoxy, methylamino, ethylamino, dimethylamino, diethylamino, N-methyl-N-ethylamino, acetylamino, N-methylcarbamoyl N-ethylcarbamoyl N,N-dimethylcarbamoyl, N,N-diethylcarbamoyl, N-methyl-N-ethylcarbamoyl, methylthio, ethylthio, methylsulfinyl, ethylsulfinyl, mesyl ethylsulfonyl, methoxycarbonyl, ethoxycarbonyl, N-methylsulfamoyl N-ethylsulfamoyl N,N-dimethylsulfamoyl N,N-diethylsulfamoyl, N-methyl-N-ethylsulfamoyl, carbocyclyl, aryl, or heterocyclyl;
n=1-10.

For example, n may be selected from an integer from 3 to 6.

For example, the compound may be a compound of Formula 1A:

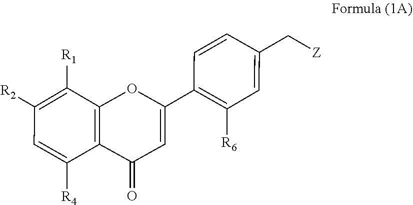

wherein:
Z is —[P(R¹¹)₃]X, wherein X is a counter anion;
R¹, R², and R⁴, independently, are selected from —OH, —O—C₁₋₄ alkyl, —OC(O)R₁₃, —OC(O)NHR¹³, —OC(O)N(R¹³)₂; or R¹ and R² together form —O—(C₁₋₃ alkylene)-O—;
R⁶ is selected from H; halo; —CN; —NO₂; —R^{β}; —OH, —OR^{β}; —SH; —SR^{β}; —SOR^{β}; —SO₂H; —SO₂R^{β}; —SO₂NH₂; —SO₂NHR^{β}; —SO₂N(R^{β})₂; —NH₂; —NHR^{β}; —N(R^{β})₂; —CHO; —COR^{β}; —COOH; —COOR^{β}; —OCOR^{β}; and benzyl optionally substituted with 1-3 —R^{β};
each —R^{β} is independently selected from a C₁-C₆ alkyl, C₂-C₆ alkenyl, C₂-C₆ alkynyl or C₃-C₁₄ cyclic group, and wherein any —R^{β} may optionally be substituted with one or more C₁-C₄ alkyl, C₁-C₄ haloalkyl, C₃-C₇ cycloalkyl, —O(C₁-C₄ alkyl), —O(C₁-C₄ haloalkyl), —O(C₃-C₇ cycloalkyl), halo, —OH, —NH₂, —CN, —NO₂, —C≡CH, —CHO, —CON(CH₃)₂ or oxo (═O) groups;
each —R¹¹ is independently selected from H, C₁-C₆ alkyl, C₂-C₆ alkenyl, C₃-C₁₄ aryl group, or C₃-C₁₄ aliphatic cyclic group, and wherein any —R¹¹ may optionally be substituted with one or more C₁-C₄ alkyl, C₁-C₄ haloalkyl, C₃-C₇ cycloalkyl, —O(C₁-C₄ alkyl), —O(C₁-C₄ haloalkyl), —O(C₃-C₇ cycloalkyl), halo, —OH, —NH₂, —CN, —C≡CH or oxo (═O) groups
each —R¹³ is independently selected from a H, C₁-C₆ alkyl, C₂-C₆ alkenyl, C₂-C₆ alkynyl, C₃₋₁₄ cyclic group, halo, —NO₂, —CN, —OH, —NH₂, mercapto, formyl, carboxy, carbamoyl, C₁₋₆ alkoxy, C₁₋₆ alkylthio, —NH(C₁₋₆ alkyl), —N(C₁₋₆ alkyl)₂, C₁₋₆ alkylsulfinyl, C₁₋₆ alkylsulfonyl, or arylsulfonyl, wherein any —R¹³ may optionally be substituted with one or more —R¹⁴;
each R¹⁴ is independently selected from a C₁-C₆ alkyl, C₂-C₆ alkenyl, C₂-C₆ alkynyl, C₃₋₁₄ cyclic group, halo, —NO₂, —CN, —OH, —NH₂, mercapto, formyl, carboxy, carbamoyl, C₁₋₆ alkoxy, C₁₋₆ alkylthio, —NH(C₁₋₆ alkyl), —N(C₁₋₆ alkyl)₂, C₁₋₆ alkylsulfinyl, C₁₋₆ alkylsulfonyl, or arylsulfonyl, wherein any —R₁₄ may optionally be substituted with one or more —R₁₅;
each —R¹⁵ is independently selected from halogen, nitro, cyano, hydroxy, trifluoromethoxy, trifluoromethyl, amino, formyl, carboxy, carbamoyl, mercapto, sulfamoyl, methyl, ethyl, methoxy, ethoxy, acetyl, acetoxy, methylamino, ethylamino, dimethylamino, diethylamino, N-methyl-N-ethylamino, acetylamino, N-methylcarbamoyl N-ethylcarbamoyl N,N-dimethylcarbamoyl, N,N-diethylcarbamoyl, N-methyl-N-ethylcarbamoyl, methylthio, ethylthio, methylsulfinyl, ethylsulfinyl, mesyl ethylsulfonyl, methoxycarbonyl, ethoxycarbonyl, N-methylsulfamoyl N-ethylsulfamoyl N,N-dimethylsulfamoyl N,N-diethylsulfamoyl, N-methyl-N-ethylsulfamoyl, carbocyclyl, aryl, or heterocyclyl;

n=1-10. For example, n may be selected from an integer between 3 and 6.

For completeness, when R¹ and R² together form —O—(C₁₋₃ alkylene)-O—; R⁴ is selected from —OH, —O—C₁₋₄ alkyl, —OC(O)R₁₃, —OC(O)NHR¹³, —OC(O)N(R¹³)₂.

A second aspect of the invention provides a compound selected from the group consisting of:

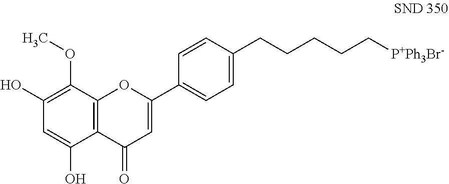

A third aspect of the invention provides pharmaceutically acceptable multi-salt, solvate or prodrug of the compound of the first or second aspect of the invention.

A fourth aspect of the invention provides a pharmaceutical composition comprising a compound of the first or second aspect of the invention, or a pharmaceutically acceptable multi-salt, solvate or prodrug of the third aspect of the invention, and a pharmaceutically acceptable excipient.

A fifth aspect of the invention provides a compound of the first or second aspect of the invention, or a pharmaceutically acceptable multi-salt, solvate or prodrug of the third aspect of the invention, or a pharmaceutical composition of the fourth aspect of the invention, for use in medicine, and/or for use in the treatment or prevention of a disease, disorder or condition. In one embodiment, the disease, disorder or condition is cancer.

A sixth aspect of the invention provides the use of a compound of the first or second aspect, a pharmaceutically effective multi-salt, solvate or prodrug of the third aspect, or a pharmaceutical composition according to the fourth aspect, in the manufacture of a medicament for the treatment or prevention of a disease, disorder or condition. Typically the treatment or prevention comprises the administration of the compound, multi-salt, solvate, prodrug or pharmaceutical composition to a subject. In one embodiment, the disease, disorder or condition is cancer.

A seventh aspect of the invention provides a method of treatment or prevention of a disease, disorder or condition, the method comprising the step of administering an effective amount of a compound of the first or second aspect, or a pharmaceutically acceptable multi-salt, solvate or prodrug of the third aspect, or a pharmaceutical composition of the fourth aspect, to thereby treat or prevent the disease, disorder or condition. Typically the administration is to a subject in need thereof. In one embodiment, the disease, disorder or condition is cancer.

### DETAILED DESCRIPTION OF THE INVENTION

A first aspect of the invention provides a compound of formula (I):

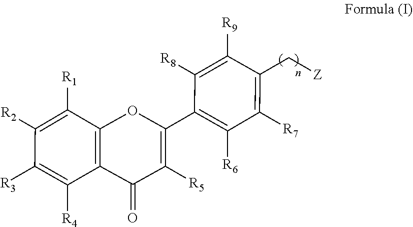

wherein:
Z is —[P(R¹¹)₃]X, wherein X is a counter anion;
R¹, R², and R⁴, independently, are selected from —OH, —O—C₁₋₄ alkyl, —OC(O)R₁₃, —OC(O)NHR¹³, —OC(O)N(R¹³)₂;
wherein R¹ and R² may together form —O—(C₁₋₃ alkylene)-O—;
R³, R⁵, R⁶, R⁷, R⁸, and R⁹, independently, are selected from H; halo; —CN; —NO₂; —R^{β}; —OH, —OR^{β}; —SH; —SR^{β}; —SOR^{β}; —SO₂H; —SO₂R^{β}; —SO₂NH₂; —SO₂NHR^{β}; —SO₂N(R^{β})₂; —NH₂; —NHR^{β}; —N(R^{β})₂; —CHO; —COR^{β}; —COOH; —COOR^{β}; —OCOR^{β}; and benzyl optionally substituted with 1-3 —R^{β};
each —R^{β} is independently selected from a C₁-C₆ alkyl, C₂-C₆ alkenyl, C₂-C₆ alkynyl or C₃-C₁₄ cyclic group, and wherein any —R^{β} may optionally be substituted with one or more C₁-C₄ alkyl, C₁-C₄ haloalkyl, C₃-C₇ cycloalkyl, —O(C₁-C₄ alkyl), —O(C₁-C₄ haloalkyl), —O(C₃-C₇ cycloalkyl), halo, —OH, —NH₂, —CN, —NO₂, —C≡CH, —CHO, —CON(CH₃)₂ or oxo (═O) groups;
each —R¹¹ is independently selected from H, C₁-C₆ alkyl, C₂-C₆ alkenyl, C₃-C₁₄ aryl group, or C₃-C₁₄ aliphatic cyclic group, and wherein any —R¹¹ may optionally be substituted with one or more C₁-C₄ alkyl, C₁-C₄ haloalkyl, C₃-C₇ cycloalkyl, —O(C₁-C₄ alkyl), —O(C₁-C₄ haloalkyl), —O(C₃-C₇ cycloalkyl), halo, —OH, —NH₂, —CN, —C≡CH or oxo (═O) groups
each —R¹³ is independently selected from a H, C₁-C₆ alkyl, C₂-C₆ alkenyl, C₂-C₆ alkynyl, C₃₋₁₄ cyclic group, halo, —NO₂, —CN, —OH, —NH₂, mercapto, formyl, carboxy, carbamoyl, C₁₋₆ alkoxy, C₁₋₆ alkylthio, —NH(C₁₋₆ alkyl), —N(C₁₋₆ alkyl)₂, C₁₋₆ alkylsulfinyl, C₁₋₆ alkylsulfonyl, or arylsulfonyl, wherein any —R¹³ may optionally be substituted with one or more —R¹⁴;
each R¹⁴ is independently selected from a C₁-C₆ alkyl, C₂-C₆ alkenyl, C₂-C₆ alkynyl, C₃₋₁₄ cyclic group, halo, —NO₂, —CN, —OH, —NH₂, mercapto, formyl, carboxy, carbamoyl, C₁₋₆ alkoxy, C₁₋₆ alkylthio, —NH(C₁₋₆ alkyl), —N(C₁₋₆ alkyl)₂, C₁₋₆ alkylsulfinyl, C₁₋₆ alkylsulfonyl, or arylsulfonyl, wherein any —R₁₄ may optionally be substituted with one or more —R₁₅;
each —R¹⁵ is independently selected from halogen, nitro, cyano, hydroxy, trifluoromethoxy, trifluoromethyl, amino, formyl, carboxy, carbamoyl, mercapto, sulfamoyl, methyl, ethyl, methoxy, ethoxy, acetyl, acetoxy, methylamino, ethylamino, dimethylamino, diethylamino, N-methyl-N-ethylamino, acetylamino, N-methylcarbamoyl N-ethylcarbamoyl N,N-dimethylcarbamoyl, N,N-diethylcarbamoyl, N-methyl-N-ethylcarbamoyl, methylthio, ethylthio, methylsulfinyl, ethylsulfinyl, mesyl ethylsulfonyl, methoxycarbonyl, ethoxycarbonyl, N-methylsulfamoyl N-ethylsulfamoyl N,N-dimethylsulfamoyl N,N-diethylsulfamoyl, N-methyl-N-ethylsulfamoyl, carbocyclyl, aryl, or heterocyclyl;
n=1-10.

In one embodiment, n=3-6.

In one embodiment, n is 3, 4, 5 or 6.

In one embodiment, n is 4, 5 or 6.

In one embodiment, n is 3 or 4.

In one embodiment, n is 4 or 5.

In one embodiment, R³, R⁵, R⁷, R⁸, and R⁹ are H; and R⁶ is selected from H; halo; —CN; —NO₂; —R^{β}; —OH, —OR^{β}; —SH; —SR; —SOR^{β}; —SO₂H; —SO₂R^{β}; —SO₂NH₂; —SO₂NHR^{β}; —SO₂N(R^{β})₂; —NH₂; —NHR^{β}; —N(R^{β})₂; —CHO; —COR^{β}; —COOH; —COOR^{β}; —OCOR^{β}; and benzyl optionally substituted with 1-3 —R^{β}. This corresponds to a compound of formula (1A):

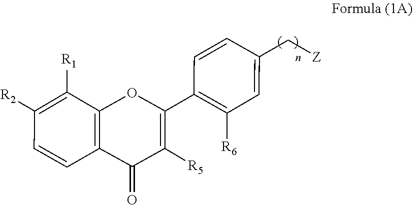

wherein R¹, R², R⁵, R⁶ and Z are as defined herein.

In one embodiment, R¹, R², and R⁴, independently, are selected from —OH, —O—C₁₋₄ alkyl, —OC(O)R₁₃, —OC(O)NHR¹³, and —OC(O)N(R¹³)₂; wherein R¹ and R² together may form —O—(C₁₋₃ alkylene)-O—; and R³, R⁵, R⁶, R⁷, R⁸, and R⁹, independently, are selected from H; halo; —CN; —NO₂; —R^{β}; —SH; —SR^{β}; —SOR^{β}; —SO₂H; —SO₂R^{β}; —SO₂NH₂; —SO₂NHR^{β}; —SO₂N(R^{β})₂; —NH₂; —NHR^{β}; —N(R^{β})₂; —CHO; —COR^{β}; —COOH; —COOR^{β}; —OCOR^{β}; and benzyl optionally substituted with 1-3 —R^{β}.

In one embodiment, R¹, R², and R⁴, independently, are selected from —OH, —O—C₁₋₄ alkyl, —OC(O)R₁₃, —OC(O)NHR¹³, and —OC(O)N(R¹³)₂; and R³, R⁵, R⁶, R⁷, R⁸, and R⁹, independently, are selected from H; halo; —CN; —NO₂; —R^{β}; —SH; —SR^{β}; —SOR^{β}; —SO₂H; —SO₂R^{β}; —SO₂NH₂; —SO₂NHR^{β}; —SO₂N(R^{β})₂; —NH₂; —NHR^{β}; —N(R^{β})₂; —CHO; —COR^{β}; —COOH; —COOR^{β}; —OCOR^{β}; and benzyl optionally substituted with 1-3 —R^{β}.

In one embodiment, R¹, R², and R⁴, independently, are selected from —OH, —O—C₁₋₄ alkyl, —OC(O)R₁₃, —OC(O)NHR¹³, and —OC(O)N(R¹³)₂; wherein R¹ and R² together may form —O—(C₁₋₃ alkylene)-O—.

In one embodiment, R¹, R², and R⁴, independently, are selected from —OH, —O—C₁₋₄ alkyl, —OC(O)R₁₃, —OC(O)NHR¹³, and —OC(O)N(R¹³)₂.

In one embodiment, R¹, R², and R⁴, independently, are selected from —OH, —O—C₁₋₄ alkyl, and —OC(O)R₁₃.

In one embodiment, R¹, R², and R⁴, independently, are selected from —OH, —OCH₃, —OC(O)C(CH₃)₃, —OC(O)NH—C₁₋₃ alkyl, and —OC(O)N(CH₃)₂, or R¹ and R² together form —O—CH₂—O—.

In one embodiment, R¹, R², and R⁴, independently, are selected from —OH, —OCH₃, —OC(O)C(CH₃)₃, —OC(O)NH—C₁₋₃ alkyl, and —OC(O)N(CH₃)₂.

In one embodiment, R¹, R², and R⁴, independently, are selected from —OH, and —O—C₁₋₄ alkyl. For example, R¹, R², and R⁴, independently, are selected from —OH, and —O—C₁₋₃ alkyl. For example, R¹, R², and R⁴, independently, are selected from —OH, and —O—C₁₋₂ alkyl. For example, R¹, R², and R⁴, independently, are selected from —OH and —O—CH₃.

In one embodiment, R¹ and R², independently, are selected from —OH, —O—C₁₋₄ alkyl, —OC(O)R₁₃, —OC(O)NHR¹³, —OC(O)N(R¹³)₂; and R⁴ is —OH.

In one embodiment, R¹ and R², independently, are selected from —OH and —O—C₁₋₄ alkyl; and R⁴ is —OH.

In one embodiment, R¹ and R², independently, are selected from —OH and —OCH₃; and R⁴ is —OH.

In one embodiment, R¹ and R⁴, independently, are selected from —OH, —O—C₁₋₄ alkyl, —OC(O)R₁₃, —OC(O)NHR¹³, —OC(O)N(R¹³)₂; and R² is —OH.

In one embodiment, R¹ and R⁴, independently, are selected from —OH and —O—C₁₋₄ alkyl; and R² is —OH.

In one embodiment, R¹ and R⁴, independently, are selected from —OH and —OCH₃; and R² is —OH.

In one embodiment, R¹ is selected from —OH, —O—C₁₋₄ alkyl, —OC(O)R₁₃, —OC(O)NHR¹³, —OC(O)N(R¹³)₂; and R² and R⁴ are —OH.

In one embodiment, R¹ is selected from —OH, and —O—C₁₋₄ alkyl; and R² and R⁴ are —OH.

In one embodiment, R¹ is selected from OH and —OCH₃; and R² and R⁴ are —OH.

In one embodiment, R³, R⁵, R⁶, R⁷, R⁸, and R⁹, independently, are selected from H; halo; —CN; —NO₂; —R^{β}; —OH, —OR^{β}; —SH; —SR^{β}; —SOR^{β}; —SO₂H; —SO₂R^{β}; —SO₂NH₂; —SO₂NHR^{β}; —SO₂N(R^{β})₂; —NH₂; —NHR^{β}; —N(R^{β})₂; —CHO; —COR^{β}; —COOH; —COOR^{β}; —OCOR^{β}; and benzyl optionally substituted with 1-3 —R^{β}.

In one embodiment, R³, R⁵, R⁶, R⁷, R⁸, and R⁹, independently, are selected from H; halo; —CN; —NO₂; —R^{β}; —SH; —SR^{β}; —SOR^{β}; —SO₂H; —SO₂R^{β}; —SO₂NH₂; —SO₂NHR^{β}; —SO₂N(R^{β})₂; —NH₂; —NHR^{β}; —N(R^{β})₂; —CHO; —COR^{β}; —COOH; —COOR^{β}; and benzyl optionally substituted with 1-3 —R^{β}.

In one embodiment, R³, R⁵, R⁶, R⁷, R⁸, and R⁹, independently, are selected from H; halo; —CN; —NO₂; —R^{β}; —SH; —SR^{β}; —SOR^{β}; —SO₂H; —SO₂R^{β}; —SO₂NH₂; —SO₂NHR^{β}; —SO₂N(R^{β})₂; —NH₂; —NHR^{β}; —N(R^{β})₂; —CHO; —COR^{β}; —COOH; —COOR^{β}; and benzyl optionally substituted with 1-3 —R^{β}.

In one embodiment, R³, R⁵, R⁶, R⁷, R⁸, and R⁹, independently, are selected from H; halo; —CN; —NO₂; —R^{β}; —NH₂; —NHR^{β}; —N(R^{β})₂; —CHO; —COR^{β}; —COOH; —COOR^{β}; and —OCOR^{β}.

In one embodiment, R³, R⁵, R⁶, R⁷, R⁸, and R⁹, independently, are selected from H; halo; —CN; —NO₂; —R^{β}; —NH₂; —NHR^{β}; —N(R^{β})₂; —CHO; —COR^{β}; —COOH; and —COOR^{β}.

In one embodiment, R³, R⁵, R⁶, R⁷, R⁸, and R⁹, independently, are selected from H; halo; —CN; —NO₂; —R^{β}; —NH₂; —NHR^{β}; —N(R^{β})₂; and —CHO.

In one embodiment, R³, R⁵, R⁶, R⁷, R⁸, and R⁹, independently, are selected from H; halo; —CN; —NO₂; —SH; —SO₂H; and —NH₂.

In one embodiment, R³, R⁵, R⁶, R⁷, R⁸, and R⁹ are H.

In one embodiment, R¹, R², and R⁴, independently, are selected from —OH, —O—C₁₋₄ alkyl, —OC(O)R₁₃, —OC(O)NHR¹³, —OC(O)N(R¹³)₂; and R³, R⁵, R⁶, R⁷, R⁸, and R⁹, independently, are selected from H; halo; —CN; —NO₂; —R^{β}; —OH, —OR^{β}; —SH; —SR^{β}; —SOR^{β}; —SO₂H; —SO₂R^{β}; —SO₂NH₂; —SO₂NHR^{β}; —SO₂N(R^{β})₂; —NH₂; —NHR^{β}; —N(R^{β})₂; —CHO; —COR^{β}; —COOH; —COOR^{β}; —OCOR^{β}; and benzyl optionally substituted with 1-3 —R^{β}. For example, R¹, R², and R⁴, independently, are selected from —OH, —OCH₃, —OC(O)C(CH₃)₃, —OC(O)NH—C₁₋₃ alkyl, and —OC(O)N(CH₃)₂; and R³, R⁵, R⁶, R⁷, R⁸, and R⁹, independently, are selected from H; halo; —CN; —NO₂; —SH; —SO₂H; and —NH₂. For example, R¹, R², and R⁴, independently, are selected from —OH, —OCH₃, —OC(O)C(CH₃)₃, —OC(O)NH—C₁₋₃ alkyl, and —OC(O)N(CH₃)₂; and R³, R⁵, R⁶, R⁷, R⁸, and R⁹ are H.

In one embodiment, R¹, R², and R⁴, independently, are selected from —OH, —O—C₁₋₄ alkyl, —OC(O)R₁₃, —OC(O)NHR¹³, —OC(O)N(R¹³)₂; and R³, R⁵, R⁶, R⁷, R⁸, and R⁹, independently, are selected from H; halo; —CN; —NO₂; —R^{β}; —OH, —OR^{β}; —SH; —SR^{β}; —SOR^{β}; —SO₂H; —SO₂R^{β}; —SO₂NH₂; —SO₂NHR^{β}; —SO₂N(R^{β})₂; —NH₂; —NHR^{β}; —N(R^{β})₂; —CHO; —COR^{β}; —COOH; —COOR^{β}; —OCOR^{β}; and benzyl optionally substituted with 1-3 —R^{β}. For example, R¹, R², and R⁴, independently, are selected from —OH, and —OCH₃; and R³, R⁵, R⁶, R⁷, R⁸, and R⁹, independently, are selected from H; halo; —CN; —NO₂; —SH; —SO₂H; and —NH₂. For example, R¹, R², and R⁴, independently, are selected from —OH, and —OCH₃; and R³, R⁵, R⁶, R⁷, R⁸, and R⁹ are H.

In one embodiment, R¹ and R² together form a —O—(C₁₋₃ alkylene)-O— group. For example, R¹ and R² together form —O-(methylene)-O—.

In one embodiment, each —R^{β} is independently selected from a C₁-C₆ alkyl, C₂-C₆ alkenyl, C₂-C₆ alkynyl or C₃-C₁₄ cyclic group, and wherein any —R^{β} may optionally be substituted with one or more C₁-C₄ alkyl, C₁-C₄ haloalkyl, C₃-C₇ cycloalkyl, —O(C₁-C₄ alkyl), —O(C₁-C₄ haloalkyl), —O(C₃-C₇ cycloalkyl), halo, —OH, —NH₂, —CN, —NO₂, —C≡CH, —CHO, —CON(CH₃)₂ or oxo (═O) groups.

In one embodiment, R^{β} is independently selected from a C₁-C₆ alkyl, C₂-C₆ alkenyl, C₂-C₆ alkynyl or C₃-C₁₄ cyclic group, and wherein any —R^{β} may optionally be substituted with one or more halo, —OH, —NH₂, —CN, —NO₂, —C≡CH, —CHO, —CON(CH₃)₂ or oxo (═O) groups.

In one embodiment, each —R^{β} is independently selected from a C₁-C₆ alkyl, C₂-C₆ alkenyl, C₂-C₆ alkynyl or C₃-C₁₄ cyclic group.

In one embodiment, each —R^{β} is independently selected from —CF₃ and —CHF₂.

In one embodiment, each —R^{β} is independently selected from a methyl, ethyl, n-propyl, i-propyl, n-butyl, i-butyl, t-butyl, n-pentyl, ethenyl, propenyl, 1-butenyl, 2-butenyl, 1-pentenyl, 1-hexenyl, 1,3-butadienyl, 1,3-pentadienyl, 1,4-pentadienyl, 1,4-hexadienyl, ethynyl, propargyl, but-1-ynyl or but-2-ynyl group.

In one embodiment, each —R^{β} is independently selected from a methyl, ethyl, n-propyl, i-propyl, n-butyl, i-butyl, t-butyl, or n-pentyl group.

In one embodiment, X is a pharmaceutically acceptable counter anion.

In one embodiment, X is selected from but not limited to halides (for example fluoride, chloride, bromide or iodide) or other inorganic anions (for example nitrate, perchlorate, sulfate, bisulfate, or phosphate) or organic anions (for example propanoate, butyrate, glycolate, lactate, mandelate, citrate, acetate, benzoate, salicylate, succinate, malate, tartrate, fumarate, maleate, hydroxymaleate, galactarate, gluconate, pantothenate, pamoate, methanesulfonate, trifluoromethanesulfonate, ethanesulfonate, 2-hydroxyethanesulfonate, benzenesulfonate, toluene-p-sulfonate, naphthalene-2-sulfonate, camphorsulfonate, ornithinate, glutamate or aspartate).

In one embodiment, X may be a fluoride, chloride, bromide or iodide.

In one embodiment, X is bromide or chloride.

In one embodiment, X is bromide.

In one embodiment, Z is —[P(R¹¹)₃]X, wherein each —R¹¹ is independently selected from H, C₁-C₆ alkyl, C₂-C₆ alkenyl, C₃-C₁₄ aryl group, or C₃-C₁₄ aliphatic cyclic group, and wherein any —R¹¹ may optionally be substituted with one or more C₁-C₄ alkyl, C₁-C₄ haloalkyl, C₃-C₇ cycloalkyl, —O(C₁-C₄ alkyl), —O(C₁-C₄ haloalkyl), —O(C₃-C₇ cycloalkyl), halo, —OH, —NH₂, —CN, —C≡CH or oxo (═O) groups; and wherein X is a counter anion.

For example, X may be bromide or chloride.

In one embodiment, Z is —[P(R¹¹)₃]X, wherein each —R¹¹ is independently selected from H, C₁-C₆ alkyl, C₂-C₆ alkenyl, C₃-C₁₄ aryl group, or C₃-C₁₄ aliphatic cyclic group; and wherein X is a counter anion. For example, X may be bromide or chloride.

In one embodiment, Z is —[P(R¹¹)₃]X, wherein each —R¹¹ is independently selected from H, C₁-C₆ alkyl, C₂-C₆ alkenyl, C₃-C₁₄ aryl group, or C₃-C₁₄ aliphatic cyclic group, and wherein any —R¹¹ may optionally be substituted with one or more C₁-C₄ alkyl, C₁-C₄ haloalkyl, C₃-C₇ cycloalkyl, —O(C₁-C₄ alkyl), —O(C₁-C₄ haloalkyl), —O(C₃-C₇ cycloalkyl), halo, —OH, —NH₂, —CN, —C≡CH or oxo (═O) groups; and wherein X is a counter anion. For example, X may be bromide or chloride.

In one embodiment, Z is —[P(R¹¹)₃]X, wherein each —R¹¹ is independently selected from H, C₁-C₆ alkyl, C₂-C₆ alkenyl, C₃-C₁₄ aryl group, or C₃-C₁₄ aliphatic cyclic group; and wherein X is a counter anion. For example, X may be bromide or chloride.

In one embodiment, Z is —[P(R¹¹)₃]X, wherein each —R¹¹ is independently selected from H, C₁-C₆ alkyl, C₂-C₆ alkenyl, C₃-C₁₄ aryl group, or C₃-C₁₄ aliphatic cyclic group; and wherein X is a counter anion. For example, X may be bromide or chloride.

In one embodiment, Z is —[P(R¹¹)₃]X, wherein each —R¹¹ is independently selected from H, or C₁-C₆ alkyl, or C₃-C₁₄ aryl group; and wherein X is a counter anion. For example, X may be bromide or chloride.

In one embodiment, Z is —[P(R¹¹)₃]X, wherein each —R¹¹ is independently a C₃-C₁₄ aryl group; and wherein any —R¹¹ may optionally be substituted with one or more C₁-C₄ alkyl, halo, —OH, —NH₂, —CN, —C≡CH or oxo (═O) groups; and wherein X is a counter anion. For example, X may be bromide or chloride.

In one embodiment, two of the R¹¹ groups are the same. In one embodiment, each R¹¹ group is the same.

In one embodiment, each R¹¹ group is the same; preferably each R¹¹ is a phenyl group.

In one embodiment, Z is —[P(R¹¹)₃]X, wherein each —R¹¹ is a phenyl group; each phenyl group may optionally be substituted with one or more C₁-C₄ alkyl, halo, —OH, —NH₂, —CN, —C≡CH or oxo (═O) groups; and wherein X is a counter anion. For example, X may be bromide or chloride.

In one embodiment, each R¹¹ is a phenyl group.

In one embodiment, Z is —[P(Ph)₃]X, wherein X is a counter anion. For example, X may be bromide or chloride, or X may be bromide.

In one embodiment, each —R¹³ is independently selected from a C₁-C₆ alkyl, C₂-C₆ alkenyl, C₂-C₆ alkynyl, C₃₋₁₄ cyclic group, halo, —NO₂, —CN, —OH, —NH₂, mercapto, formyl, carboxy, carbamoyl, C₁₋₆ alkoxy, C₁₋₆ alkylthio, —NH(C₁₋₆ alkyl), —N(C₁₋₆ alkyl)₂, C₁₋₆ alkylsulfinyl, C₁₋₆ alkylsulfonyl, or arylsulfonyl, wherein any —R¹³ may optionally be substituted with one or more —R¹⁴.

In one embodiment, each —R¹³ is independently selected from a C₁-C₆ alkyl, C₂-C₆ alkenyl, C₂-C₆ alkynyl, C₃₋₁₄ cyclic group, halo, —NO₂, —CN, —OH, —NH₂, mercapto, formyl, carboxy, carbamoyl, C₁₋₆ alkoxy, C₁₋₆ alkylthio, —NH(C₁₋₆ alkyl), —N(C₁₋₆ alkyl)₂, C₁₋₆ alkylsulfinyl, C₁₋₆ alkylsulfonyl, or arylsulfonyl.

In one embodiment, each —R¹³ is independently selected from C₁₋₄ alkyl. For example, R¹³ is independently selected from C₁₋₃ alkyl.

In one embodiment, each —R¹³ is independently selected from a H, methyl, ethyl, n-propyl, i-propyl, n-butyl, i-butyl, t-butyl, n-pentyl, ethenyl, propenyl, 1-butenyl, 2-butenyl, 1-pentenyl, 1-hexenyl, 1,3-butadienyl, 1,3-pentadienyl, 1,4-pentadienyl, 1,4-hexadienyl, ethynyl, propargyl, but-1-ynyl or but-2-ynyl group.

In one embodiment, each —R¹³ is independently selected from H, methyl, ethyl, n-propyl, i-propyl, n-butyl, i-butyl, t-butyl, or n-pentyl group.

In one embodiment, each —R¹³ is independently selected from H, methyl, ethyl, propyl, and butyl.

In one embodiment, each R¹⁴ is independently selected from a C₁-C₆ alkyl, C₂-C₆ alkenyl, C₂-C₆ alkynyl, C₃₋₁₄ cyclic group, halo, —NO₂, —CN, —OH, —NH₂, mercapto, formyl, carboxy, carbamoyl, C₁₋₆ alkoxy, C₁₋₆ alkylthio, —NH(C₁₋₆ alkyl), —N(C₁₋₆ alkyl)₂, C₁₋₆ alkylsulfinyl, C₁₋₆ alkylsulfonyl, or arylsulfonyl, wherein any —R₁₄ may optionally be substituted with one or more —R₁₅.

In one embodiment, each R¹⁴ is independently selected from a halo, —NO₂, —CN, —OH, —NH₂, mercapto, formyl, carboxy, or carbamoyl group.

In one embodiment, each —R¹⁴ is independently selected from methyl, ethyl, n-propyl, i-propyl, n-butyl, i-butyl, t-butyl, n-pentyl, ethenyl, propenyl, 1-butenyl, 2-butenyl, 1-pentenyl, 1-hexenyl, 1,3-butadienyl, 1,3-pentadienyl, 1,4-pentadienyl, 1,4-hexadienyl, ethynyl, propargyl, but-1-ynyl or but-2-ynyl.

In one embodiment, each —R¹⁴ is independently selected from a methyl, ethyl, n-propyl, i-propyl, n-butyl, i-butyl, t-butyl, or n-pentyl group.

In one embodiment, each —R¹⁵ is independently selected from halogen, nitro, cyano, hydroxy, trifluoromethoxy, trifluoromethyl, amino, formyl, carboxy, carbamoyl, mercapto, sulfamoyl, methyl, ethyl, methoxy, ethoxy, acetyl, acetoxy, methylamino, ethylamino, dimethylamino, diethylamino, N-methyl-N-ethylamino, acetylamino, N-methylcarbamoyl N-ethylcarbamoyl N,N-dimethylcarbamoyl, N,N-diethylcarbamoyl, N-methyl-N-ethylcarbamoyl, methylthio, ethylthio, methylsulfinyl, ethylsulfinyl, mesyl ethylsulfonyl, methoxycarbonyl, ethoxycarbonyl, N-methylsulfamoyl N-ethylsulfamoyl N,N-dimethylsulfamoyl N,N-diethylsulfamoyl, N-methyl-N-ethylsulfamoyl, carbocyclyl, aryl, or heterocyclyl.

In one embodiment, n is an integer from 3 to 5. In one embodiment, n is an integer from 4 to 6. In one embodiment, n is 3, 4, 5, or 6. In one embodiment, n is 3. In one embodiment, n is 4.

In one embodiment, R¹, R², and R⁴, are independently selected from —OH, —OCH₃, —OCO^{t}Bu, —OCONHCH₃, —OCONHCH₂CH₃ and —OCON(CH₃)₂, wherein R¹ and R² together may form —O—CH₂—O—; R³, R⁵, R⁶, R⁷, R⁸, and R⁹ are each H; Z is —[P(R¹¹)₃]X, wherein each —R¹¹ is a phenyl group; each phenyl group may optionally be substituted with one or more C₁-C₄ alkyl, halo, —OH, —NH₂, —CN, —C≡CH or oxo (═O) groups; X is a counter anion; and n is 3 or 4. For example, X may be bromide or chloride, or X may be bromide.

In one embodiment, R¹, R², and R⁴, are independently selected from —OH, —OCH₃, —OCO^{t}Bu, —OCONHCH₃, —OCONHCH₂CH₃ and —OCON(CH₃)₂, wherein R¹ and R² together may form —O—CH₂—O—; R³, R⁵, R⁶, R⁷, R⁸, and R⁹ are each H; Z is —[P(R¹¹)₃]X, wherein each —R¹¹ is a phenyl group; each phenyl group may optionally be substituted with one or more C₁-C₄ alkyl, halo, —OH, —NH₂, —CN, —C≡CH or oxo (═O) groups; X is a counter anion; and n is 4 or 5. For example, X may be bromide or chloride, or X may be bromide.

In one embodiment, R¹, R², and R⁴, are independently selected from —OH, —OCH₃, —OCO^{t}Bu, —OCONHCH₃, —OCONHCH₂CH₃ or —OCON(CH₃)₂, wherein R¹ and R² together may form —O—CH₂—O—; R³, R⁵, R⁶, R⁷, R⁸, and R⁹ are each H; Z is —[P(Ph)₃]X; X is a counter anion; and n is 3 or 4. For example, X may be bromide or chloride, or X may be bromide.

In one embodiment, R¹, R², and R⁴, are independently selected from —OH, —OCH₃, —OCO^{t}Bu, —OCONHCH₃, —OCONHCH₂CH₃ or —OCON(CH₃)₂, wherein R¹ and R² together may form —O—CH₂—O—; R³, R⁵, R⁶, R⁷, R⁸, and R⁹ are each H; Z is —[P(Ph)₃]X; X is a counter anion; and n is 4 or 5. For example, X may be bromide or chloride, or X may be bromide.

In one embodiment, the compounds include a quaternary phosphonium group and X is a counter anion. Preferably, the counter anion X may be any pharmaceutically acceptable, non-toxic counter ion. For example, X may be bromide or chloride, or X may be bromide.

The counter anion may optionally be singly, doubly or triply charged. As the quaternary group is singly charged, if the counter anion is triply charged then the stoichiometric ratio of the quaternary group to counter anion will typically be 3:1 and if the counter anion is doubly charged then the stoichiometric ratio of the quaternary group to counter anion will typically be 2:1. If both the quaternary group and the counter anion are singly charged then the stoichiometric ratio of the quaternary group to counter anion will typically be 1:1.

In one embodiment, the counter anion will be a singly charged anion. Suitable anions X include but are not limited to halides (for example fluoride, chloride, bromide or iodide) or other inorganic anions (for example nitrate, perchlorate, sulfate, bisulfate, or phosphate) or organic anions (for example propanoate, butyrate, glycolate, lactate, mandelate, citrate, acetate, benzoate, salicylate, succinate, malate, tartrate, fumarate, maleate, hydroxymaleate, galactarate, gluconate, pantothenate, pamoate, methanesulfonate, trifluoromethanesulfonate, ethanesulfonate, 2-hydroxyethanesulfonate, benzenesulfonate, toluene-p-sulfonate, naphthalene-2-sulfonate, camphorsulfonate, ornithinate, glutamate or aspartate). The counter anion may be fluoride, chloride, bromide or iodide. For example, X may be bromide or chloride, or X may be bromide.

In one aspect of any of the above embodiments, the compound of formula (I) has a molecular weight of from 250 to 2,000 Da. Typically, the compound of formula (I) has a molecular weight of from 300 to 1,000 Da. Typically, the compound of formula (I) has a molecular weight of from 350 to 800 Da. More typically, the compound of formula (I) has a molecular weight of from 500 to 750 Da.

A second aspect of the invention provides a compound selected from the group consisting of:

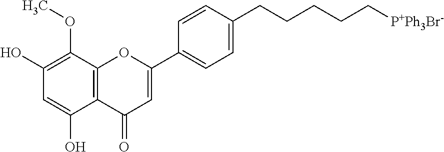

For example, the compound may be:

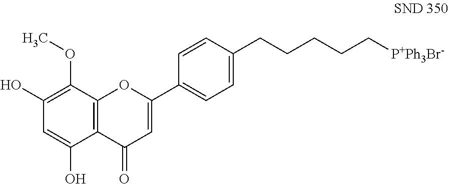

A third aspect of the invention provides a pharmaceutically acceptable multi-salt, solvate or prodrug of any compound of the first or second aspect of the invention.

The compounds of the present invention can be used both in their quaternary salt form (as a single salt). Additionally, the compounds of the present invention may contain one or more (e.g. one or two) acid addition or alkali addition salts to form a multi-salt. A multi-salt includes a quaternary salt group as well as a salt of a different group of the compound of the invention.

For the purposes of this invention, a “multi-salt” of a compound of the present invention includes an acid addition salt. Acid addition salts are preferably pharmaceutically acceptable, non-toxic addition salts with suitable acids, including but not limited to inorganic acids such as hydrohalogenic acids (for example, hydrofluoric, hydrochloric, hydrobromic or hydroiodic acid) or other inorganic acids (for example, nitric, perchloric, sulfuric or phosphoric acid); or organic acids such as organic carboxylic acids (for example, propionic, butyric, glycolic, lactic, mandelic, citric, acetic, benzoic, salicylic, succinic, malic or hydroxysuccinic, tartaric, fumaric, maleic, hydroxymaleic, mucic or galactaric, gluconic, pantothenic or pamoic acid), organic sulfonic acids (for example, methanesulfonic, trifluoromethanesulfonic, ethanesulfonic, 2-hydroxyethanesulfonic, benzenesulfonic, toluene-p-sulfonic, naphthalene-2-sulfonic or camphorsulfonic acid) or amino acids (for example, ornithinic, glutamic or aspartic acid). The acid addition salt may be a mono-, di-, tri- or multi-acid addition salt. A preferred salt is a hydrohalogenic, sulfuric, phosphoric or organic acid addition salt. A preferred salt is a hydrochloric acid addition salt.

The compounds of the present invention can be used both, in quaternary salt form and their multi-salt form. For the purposes of this invention, a “multi-salt” of a compound of the present invention includes one formed between a protic acid functionality (such as a carboxylic acid group) of a compound of the present invention and a suitable cation. Suitable cations include, but are not limited to lithium, sodium, potassium, magnesium, calcium and ammonium. The salt may be a mono-, di-, tri- or multi-salt. Preferably the salt is a mono- or di-lithium, sodium, potassium, magnesium, calcium or ammonium salt. More preferably the salt is a mono- or di-sodium salt or a mono- or di-potassium salt.

Preferably any multi-salt is a pharmaceutically acceptable non-toxic salt. However, in addition to pharmaceutically acceptable multi-salts, other salts are included in the present invention, since they have potential to serve as intermediates in the purification or preparation of other, for example, pharmaceutically acceptable salts, or are useful for identification, characterisation or purification of the free acid or base.

The compounds and/or multi-salts of the present invention may be anhydrous or in the form of a hydrate (e.g. a hemihydrate, monohydrate, dihydrate or trihydrate) or other solvate. Such solvates may be formed with common organic solvents, including but not limited to, alcoholic solvents e.g. methanol, ethanol or isopropanol.

In some embodiments of the present invention, therapeutically inactive prodrugs are provided. Prodrugs are compounds which, when administered to a subject such as a human, are converted in whole or in part to a compound of the invention. In most embodiments, the prodrugs are pharmacologically inert chemical derivatives that can be converted in vivo to the active drug molecules to exert a therapeutic effect. Any of the compounds described herein can be administered as a prodrug to increase the activity, bioavailability, or stability of the compound or to otherwise alter the properties of the compound. Typical examples of prodrugs include compounds that have biologically labile protecting groups on a functional moiety of the active compound. Prodrugs include, but are not limited to, compounds that can be oxidized, reduced, aminated, deaminated, hydroxylated, dehydroxylated, hydrolyzed, dehydrolyzed, alkylated, dealkylated, acylated, deacylated, phosphorylated, and/or dephosphorylated to produce the active compound. The present invention also encompasses multi-salts and solvates of such prodrugs as described above.

The compounds, multi-salts, solvates and prodrugs of the present invention may contain at least one chiral centre. The compounds, multi-salts, solvates and prodrugs may therefore exist in at least two isomeric forms. The present invention encompasses racemic mixtures of the compounds, multi-salts, solvates and prodrugs of the present invention as well as enantiomerically enriched and substantially enantiomerically pure isomers. For the purposes of this invention, a “substantially enantiomerically pure” isomer of a compound comprises less than 5% of other isomers of the same compound, more typically less than 2%, and most typically less than 0.5% by weight.

The compounds, multi-salts, solvates and prodrugs of the present invention may contain any stable isotope including, but not limited to ¹²C, ¹³C, ¹H, ²H (D), ¹⁴N, ¹⁵N, ¹⁶O, ¹⁷O, ¹⁸O, ¹⁹F and ¹²⁷I, and any radioisotope including, but not limited to ¹¹C, ¹⁴C, ³H (T), ¹³N, ¹⁵O, ¹⁸F, ¹²³I, ¹²⁴I, ¹²⁵I and ¹³¹I.

The compounds, multi-salts, solvates and prodrugs of the present invention may be in any polymorphic or amorphous form.

A fourth aspect of the invention provides a pharmaceutical composition comprising a compound of the first or second aspect of the invention, or a pharmaceutically acceptable multi-salt, solvate or prodrug of the third aspect of the invention, and a pharmaceutically acceptable excipient.

Conventional procedures for the selection and preparation of suitable pharmaceutical formulations are described in, for example, “Aulton's Pharmaceutics—The Design and Manufacture of Medicines”, M. E. Aulton and K. M. G. Taylor, Churchill Livingstone Elsevier, 4^{th} Ed., 2013.

Pharmaceutically acceptable excipients including adjuvants, diluents or carriers that may be used in the pharmaceutical compositions of the invention are those conventionally employed in the field of pharmaceutical formulation, and include, but are not limited to, sugars, sugar alcohols, starches, ion exchangers, alumina, aluminium stearate, lecithin, serum proteins such as human serum albumin, buffer substances such as phosphates, glycerine, sorbic acid, potassium sorbate, partial glyceride mixtures of saturated vegetable fatty acids, water, salts or electrolytes such as protamine sulfate, disodium hydrogen phosphate, potassium hydrogen phosphate, sodium chloride, zinc salts, colloidal silica, magnesium trisilicate, polyvinylpyrrolidone, cellulose-based substances, polyethylene glycol, sodium carboxymethylcellulose, polyacrylates, waxes, polyethylene-polyoxypropylene-block polymers, polyethylene glycol and wool fat.

A fifth aspect of the invention provides a compound of the first or second aspect of the invention, or a pharmaceutically acceptable multi-salt, solvate or prodrug of the third aspect of the invention, or a pharmaceutical composition of the fourth aspect of the invention, for use in medicine, and/or for use in the treatment or prevention of a disease, disorder or condition. Typically the use comprises the administration of the compound, multi-salt, solvate, prodrug or pharmaceutical composition to a subject.

An sixth aspect of the invention provides the use of a compound of the first or second aspect, a pharmaceutically effective multi-salt, solvate or prodrug of the third aspect, or a pharmaceutical composition according to the fourth aspect in the manufacture of a medicament for the treatment or prevention of a disease, disorder or condition. Typically the treatment or prevention comprises the administration of the compound, multi-salt, solvate, prodrug or pharmaceutical composition to a subject.

A seventh aspect of the invention provides a method of treatment or prevention of a disease, disorder or condition, the method comprising the step of administering an effective amount of a compound of the first or second aspect, or a pharmaceutically acceptable multi-salt, solvate or prodrug of the third aspect, or a pharmaceutical composition of the fourth aspect, to thereby treat or prevent the disease, disorder or condition. Typically the administration is to a subject in need thereof.

The term “treatment” as used herein refers equally to curative therapy, and ameliorating or palliative therapy. The term includes obtaining beneficial or desired physiological results, which may or may not be established clinically. Beneficial or desired clinical results include, but are not limited to, the alleviation of symptoms, the prevention of symptoms, the diminishment of extent of disease, the stabilisation (i.e., not worsening) of a condition, the delay or slowing of progression/worsening of a condition/symptoms, the amelioration or palliation of the condition/symptoms, and remission (whether partial or total), whether detectable or undetectable. The term “palliation”, and variations thereof, as used herein, means that the extent and/or undesirable manifestations of a physiological condition or symptom are lessened and/or time course of the progression is slowed or lengthened, as compared to not administering a compound, multi-salt, solvate, prodrug or pharmaceutical composition of the present invention. The term “prevention” as used herein in relation to a disease, disorder or condition, relates to prophylactic or preventative therapy, as well as therapy to reduce the risk of developing the disease, disorder or condition. The term “prevention” includes both the avoidance of occurrence of the disease, disorder or condition, and the delay in onset of the disease, disorder or condition. Any statistically significant avoidance of occurrence, delay in onset or reduction in risk as measured by a controlled clinical trial may be deemed a prevention of the disease, disorder or condition. Subjects amenable to prevention include those at heightened risk of a disease, disorder or condition as identified by genetic or biochemical markers. Typically, the genetic or biochemical markers are appropriate to the disease, disorder or condition under consideration and may include for example, beta-amyloid 42, tau and phosphor-tau.

In general embodiments, the disease, disorder or condition is cancer.

In one embodiment, the cancer is brain cancer, breast cancer, colon cancer, leukaemia, lymphoma, or melanoma.

In one embodiment, the cancer is breast cancer, colon cancer, lymphoma, or melanoma.

In one embodiment the cancer is brain cancer.

In one embodiment the cancer is breast cancer.

In one embodiment the cancer is colon cancer.

In one embodiment the cancer is leukaemia.

In one embodiment the cancer is lung cancer.

In one embodiment the cancer is ovarian cancer.

In one embodiment the cancer is pancreatic cancer.

In one embodiment the cancer is prostate cancer.

In one embodiment the cancer is lymphoma.

In one embodiment the cancer is skin cancer (melanoma).

Unless stated otherwise, in any aspect of the invention, the subject may be any human or other animal. Typically, the subject is a mammal, more typically a human or a domesticated mammal such as a cow, pig, lamb, goat, horse, cat, dog, etc. Most typically, the subject is a human.

Any of the medicaments employed in the present invention can be administered by oral, parental (including intravenous, subcutaneous, intramuscular, intradermal, intratracheal, intraperitoneal, intraarticular, intracranial and epidural), airway (aerosol), rectal, vaginal or topical (including transdermal, buccal, mucosal and sublingual) administration.

Typically, the mode of administration selected is that most appropriate to the disorder or disease to be treated or prevented.

For oral administration, the compounds, multi-salts, solvates or prodrugs of the present invention will generally be provided in the form of tablets, capsules, hard or soft gelatine capsules, caplets, troches or lozenges, as a powder or granules, or as an aqueous solution, suspension or dispersion.

Tablets for oral use may include the active ingredient mixed with pharmaceutically acceptable excipients such as inert diluents, disintegrating agents, binding agents, lubricating agents, sweetening agents, flavouring agents, colouring agents and preservatives. Suitable inert diluents include sodium and calcium carbonate, sodium and calcium phosphate, and lactose. Corn starch and alginic acid are suitable disintegrating agents. Binding agents may include starch and gelatine. The lubricating agent, if present, may be magnesium stearate, stearic acid or tale. If desired, the tablets may be coated with a material, such as glyceryl monostearate or glyceryl distearate, to delay absorption in the gastrointestinal tract. Tablets may also be effervescent and/or dissolving tablets.

Capsules for oral use include hard gelatine capsules in which the active ingredient is mixed with a solid diluent, and soft gelatine capsules wherein the active ingredient is mixed with water or an oil such as peanut oil, liquid paraffin or olive oil.

Powders or granules for oral use may be provided in sachets or tubs. Aqueous solutions, suspensions or dispersions may be prepared by the addition of water to powders, granules or tablets.

Any form suitable for oral administration may optionally include sweetening agents such as sugar, flavouring agents, colouring agents and/or preservatives.

Formulations for rectal administration may be presented as a suppository with a suitable base comprising, for example, cocoa butter or a salicylate.

Formulations suitable for vaginal administration may be presented as pessaries, tampons, creams, gels, pastes, foams or spray formulations containing in addition to the active ingredient such carriers as are known in the art to be appropriate.

For parenteral use, the compounds, multi-salts, solvates or prodrugs of the present invention will generally be provided in a sterile aqueous solution or suspension, buffered to an appropriate pH and isotonicity. Suitable aqueous vehicles include Ringer's solution and isotonic sodium chloride or glucose. Aqueous suspensions according to the invention may include suspending agents such as cellulose derivatives, sodium alginate, polyvinylpyrrolidone and gum tragacanth, and a wetting agent such as lecithin. Suitable preservatives for aqueous suspensions include ethyl and n-propyl p-hydroxybenzoate. The compounds of the invention may also be presented as liposome formulations.

For transdermal and other topical administration, the compounds, multi-salts, solvates or prodrugs of the invention will generally be provided in the form of ointments, cataplasms (poultices), pastes, powders, dressings, creams, plasters or patches.

Suitable suspensions and solutions can be used in inhalers for airway (aerosol) administration.

The dose of the compounds, multi-salts, solvates or prodrugs of the present invention will, of course, vary with the disorder or disease to be treated or prevented. In general, a suitable dose will be in the range of 0.01 to 500 mg per kilogram body weight of the recipient per day. The desired dose may be presented at an appropriate interval such as once every other day, once a day, twice a day, three times a day or four times a day. The desired dose may be administered in unit dosage form, for example, containing 1 mg to 50 g of active ingredient per unit dosage form.

An eighth aspect of the invention provides a method of treatment or prevention of a disease, disorder or condition, the method comprising the step of administering an effective amount of a compound according to formula (1) as defined herein, or a pharmaceutically acceptable multi-salt, solvate or prodrug thereof, to thereby treat or prevent the disease, disorder or condition. Typically the administration is to a subject in need thereof. In one embodiment, the disease, disorder or condition is cancer.

### Definitions

In the context of the present specification, a “hydrocarbyl” substituent group or a hydrocarbyl moiety in a substituent group only includes carbon and hydrogen atoms but, unless stated otherwise, does not include any heteroatoms, such as N, O or S, in its carbon skeleton. A hydrocarbyl group/moiety may be saturated or unsaturated (including aromatic), and may be straight-chained or branched, or be or include cyclic groups wherein, unless stated otherwise, the cyclic group does not include any heteroatoms, such as N, O or S, in its carbon skeleton. Examples of hydrocarbyl groups include alkyl, alkenyl, alkynyl, cycloalkyl, cycloalkenyl and aryl groups/moieties and combinations of all of these groups/moieties. Typically a hydrocarbyl group is a C₁-C₁₂ hydrocarbyl group. More typically a hydrocarbyl group is a C₁-C₁₀ hydrocarbyl group. A “hydrocarbylene” group is similarly defined as a divalent hydrocarbyl group.

An “alkyl” substituent group or an alkyl moiety in a substituent group may be linear or branched. Examples of alkyl groups/moieties include methyl, ethyl, n-propyl, i-propyl, n-butyl, i-butyl, t-butyl and n-pentyl groups/moieties. Unless stated otherwise, the term “alkyl” does not include “cycloalkyl”. Typically an alkyl group is a C₁-C₁₂ alkyl group. More typically an alkyl group is a C₁-C₆ alkyl group. An “alkylene” group is similarly defined as a divalent alkyl group.

An “alkenyl” substituent group or an alkenyl moiety in a substituent group refers to an unsaturated alkyl group or moiety having one or more carbon-carbon double bonds. Examples of alkenyl groups/moieties include ethenyl, propenyl, 1-butenyl, 2-butenyl, 1-pentenyl, 1-hexenyl, 1,3-butadienyl, 1,3-pentadienyl, 1,4-pentadienyl and 1,4-hexadienyl groups/moieties. Unless stated otherwise, the term “alkenyl” does not include “cycloalkenyl”. Typically an alkenyl group is a C₂-C₁₂ alkenyl group. More typically an alkenyl group is a C₂-C₆ alkenyl group. An “alkenylene” group is similarly defined as a divalent alkenyl group.

An “alkynyl” substituent group or an alkynyl moiety in a substituent group refers to an unsaturated alkyl group or moiety having one or more carbon-carbon triple bonds. Examples of alkynyl groups/moieties include ethynyl, propargyl, but-1-ynyl and but-2-ynyl. Typically an alkynyl group is a C₂-C₁₂ alkynyl group. More typically an alkynyl group is a C₂-C₆ alkynyl group. An “alkynylene” group is similarly defined as a divalent alkynyl group.

A “haloalkyl” substituent group or haloalkyl group in a substituent group refers to an alkyl, alkenyl, or alkynyl substituent group or moiety including one or more carbon atoms and one or more halo atoms, e.g. Cl, Br, I, or F. Each halo atom replaces a hydrogen of the alkyl, alkenyl, or alkynyl substituent group or moiety. Examples include —CH₂F —CHF₂, —CHI₂, —CHBr₂, —CHCl₂, —CF₃, —CH₂CF₃ and CF₂CH₃.

An “alkoxy” substituent group or alkoxy group in a substituent group refers to an alkyl, alkenyl, or alkynyl substituent group or moiety including one or more carbon atoms and one or more oxygen atoms. Each oxygen atom replaces a carbon atom (for example the terminal or bonding carbon) of the alkyl, alkenyl, or alkynyl substituent group or moiety. Examples include —OCH₃, —OCH₂CH₃, —OCH₂CH₂CH₃, and —OCH(CH₃)(CH₃).

An “alkylthio” substituent group or alkylthio group in a substituent group refers to an alkyl, alkenyl, or alkynyl substituent group or moiety including one or more carbon atoms and one or more sulphur atoms. Each sulphur atom replaces a carbon atom (for example the terminal or bonding carbon) of the alkyl, alkenyl, or alkynyl substituent group or moiety. Examples include —SCH₃, —SCH₂CH₃, —SCH₂CH₂CH₃, and —SCH(CH₃)(CH₃).

An “alkylsulfinyl” substituent group or alkylsulfinyl group in a substituent group refers to an alkyl, alkenyl, or alkynyl substituent group or moiety including one or more carbon atoms and one or more sulfinyl groups (—S(═O)—). Each sulfinyl group replaces a carbon atom (for example the terminal or bonding carbon) of the alkyl, alkenyl, or alkynyl substituent group or moiety. Examples include —S(═O)CH₃, —S(═O)CH₂CH₃, —S(═O)CH₂CH₂CH₃, and —S(═O)CH(CH₃)(CH₃).

An “alkylsulfonyl” substituent group or alkylsulfonyl group in a substituent group refers to an alkyl, alkenyl, or alkynyl substituent group or moiety including one or more carbon atoms and one or more sulfonyl groups (—SO₂—). Each sulfonyl group replaces a carbon atom (for example the terminal or bonding carbon) of the alkyl, alkenyl, or alkynyl substituent group or moiety. Examples include —SO₂(CH₃), —SO₂(CH₂CH₃), —SO₂(CH₂CH₂CH₃), and —SO₂(CH(CH₃)(CH₃)).

An “arylsulfonyl” substituent group or arylsulfonyl group in a substituent group refers to an aryl substituent group or moiety including one or more carbon atoms and one or more sulfonyl groups (—SO₂—). Each sulfonyl group replaces a carbon atom (for example the terminal or bonding carbon) of the alkyl, alkenyl, or alkynyl substituent group or moiety. Examples include —SO₂(CH₃), —SO₂(CH₂CH₃), —SO₂(CH₂CH₂CH₃), and —SO₂(CH(CH₃)(CH₃)).

A “cyclic” substituent group or a cyclic moiety in a substituent group refers to any hydrocarbyl ring, wherein the hydrocarbyl ring may be saturated or unsaturated and may include one or more heteroatoms, e.g. N, O or S, in its carbon skeleton. Examples of cyclic groups include aliphatic cyclic, cycloalkyl, cycloalkenyl, heterocyclic, aryl and heteroaryl groups as discussed below. A cyclic group may be monocyclic, bicyclic (e.g. bridged, fused or spiro), or polycyclic. Typically, a cyclic group is a 3- to 12-membered cyclic group, which means it contains from 3 to 12 ring atoms. More typically, a cyclic group is a 3- to 7-membered monocyclic group, which means it contains from 3 to 7 ring atoms.

A “heterocyclic” substituent group or a heterocyclic moiety in a substituent group refers to a cyclic group or moiety including one or more carbon atoms and one or more heteroatoms, e.g. N, O or S, in the ring structure. Examples of heterocyclic groups include heteroaryl groups as discussed below and non-aromatic heterocyclic groups such as azetidinyl, azetinyl, tetrahydrofuranyl, pyrrolidinyl, tetrahydrothiophenyl, tetrahydropyranyl, piperidinyl, piperazinyl, morpholinyl and thiomorpholinyl groups.

An “aliphatic cyclic” substituent group or aliphatic cyclic moiety in a substituent group refers to a hydrocarbyl cyclic group or moiety that is not aromatic. The aliphatic cyclic group may be saturated or unsaturated and may include one or more heteroatoms, e.g. N, O or S, in its carbon skeleton. Examples include cyclopropyl, cyclohexyl and morpholinyl. Unless stated otherwise, an aliphatic cyclic substituent group or moiety may include monocyclic, bicyclic or polycyclic hydrocarbyl rings.

A “cycloalkyl” substituent group or a cycloalkyl moiety in a substituent group refers to a saturated hydrocarbyl ring containing, for example, from 3 to 7 carbon atoms, examples of which include cyclopropyl, cyclobutyl, cyclopentyl and cyclohexyl. Unless stated otherwise, a cycloalkyl substituent group or moiety may include monocyclic, bicyclic or polycyclic hydrocarbyl rings.

A “cycloalkenyl” substituent group or a cycloalkenyl moiety in a substituent group refers to a non-aromatic unsaturated hydrocarbyl ring having one or more carbon-carbon double bonds and containing, for example, from 3 to 7 carbon atoms, examples of which include cyclopent-1-en-1-yl, cyclohex-1-en-1-yl and cyclohex-1,3-dien-1-yl. Unless stated otherwise, a cycloalkenyl substituent group or moiety may include monocyclic, bicyclic or polycyclic hydrocarbyl rings.

An “aryl” substituent group or an aryl moiety in a substituent group refers to an aromatic hydrocarbyl ring. The term “aryl” includes monocyclic aromatic hydrocarbons and polycyclic fused ring aromatic hydrocarbons wherein all of the fused ring systems (excluding any ring systems which are part of or formed by optional substituents) are aromatic. Examples of aryl groups/moieties include phenyl, naphthyl, anthracenyl and phenanthrenyl. Unless stated otherwise, the term “aryl” does not include “heteroaryl”.

A “heteroaryl” substituent group or a heteroaryl moiety in a substituent group refers to an aromatic heterocyclic group or moiety. The term “heteroaryl” includes monocyclic aromatic heterocycles and polycyclic fused ring aromatic heterocycles wherein all of the fused ring systems (excluding any ring systems which are part of or formed by optional substituents) are aromatic. Examples of heteroaryl groups/moieties include the following:

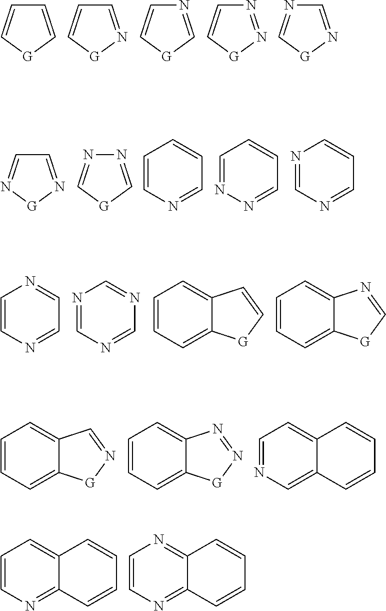

wherein G=O, S or NH.

For the purposes of the present specification, where a combination of moieties is referred to as one group, for example, arylalkyl, arylalkenyl, arylalkynyl, alkylaryl, alkenylaryl or alkynylaryl, the last mentioned moiety contains the atom by which the group is attached to the rest of the molecule. An example of an arylalkyl group is benzyl.

Typically a substituted group comprises 1, 2, 3 or 4 substituents, more typically 1, 2 or 3 substituents, more typically 1 or 2 substituents, and even more typically 1 substituent.

Unless stated otherwise, any divalent bridging substituent (e.g. —O—, —S—, —NH—, —N(R^{β})— or —R^{α}—) of an optionally substituted group or moiety must only be attached to the specified group or moiety and may not be attached to a second group or moiety, even if the second group or moiety can itself be optionally substituted.

The term “halo” includes fluoro, chloro, bromo and iodo.

Where reference is made to a carbon atom of a group being replaced by an N, O or S atom, what is intended is that:

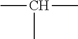

is replaced by

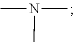

—CH₂— is replaced by —NH—, —O— or —S—;
—CH₃ is replaced by —NH₂, —OH, or —SH;
—CH═ is replaced by —N═;
CH₂═ is replaced by NH═, O═ or S═; or
CH≡ is replaced by N≡.

In the context of the present specification, unless otherwise stated, a Cₓ-C_{y} group is defined as a group containing from x to y carbon atoms. For example, a C₁-C₄ alkyl group is defined as an alkyl group containing from 1 to 4 carbon atoms. Optional substituents and moieties are not taken into account when calculating the total number of carbon atoms in the parent group substituted with the optional substituents and/or containing the optional moieties. For the avoidance of doubt, replacement heteroatoms, e.g. N, O or S, are counted as carbon atoms when calculating the number of carbon atoms in a Cₓ-C_{y} group. For example, a morpholinyl group is to be considered a C₆ heterocyclic group, not a C₄ heterocyclic group.

A “protecting group” refers to a grouping of atoms that when attached to a reactive functional group (e.g. OH) in a compound masks, reduces or prevents reactivity of the functional group.

In the context of the present specification, ═ is a double bond; ≡ is a triple bond.

The protection and deprotection of functional groups is described in ‘Protective Groups in Organic Synthesis’, 2^{nd} edition, T. W. Greene and P. G. M Wuts, Wiley-Interscience.

For the avoidance of doubt, insofar as is practicable any embodiment of a given aspect of the present invention may occur in combination with any other embodiment of the same aspect of the present invention. In addition, insofar as is practicable it is to be understood that any preferred, typical or optional embodiment of any aspect of the present invention should also be considered as a preferred, typical or optional embodiment of any other aspect of the present invention.

### EXAMPLES

The following nomenclature is used to refer to the following compounds.

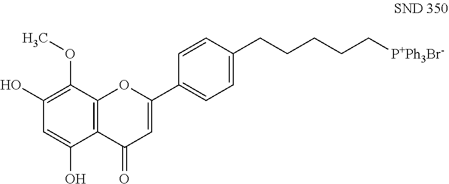

### Examples—Compound Synthesis

Compounds of the invention are synthesised employing a route of synthesis shown below. The general route of synthesis is illustrated below by reference to the synthesis of a specific compound. However, this is merely illustrative of a more general synthesis that can be employed to synthesise all compounds of the invention.

Route of Synthesis:

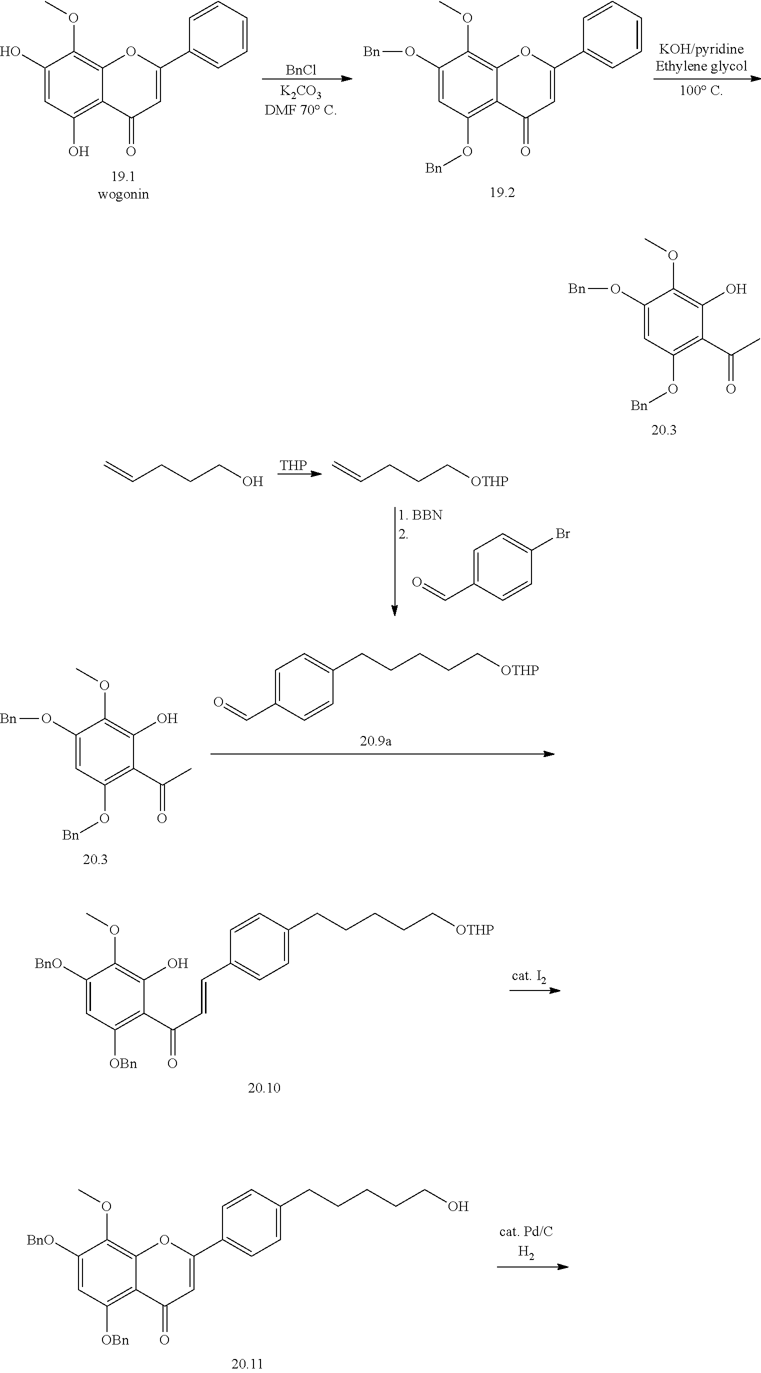

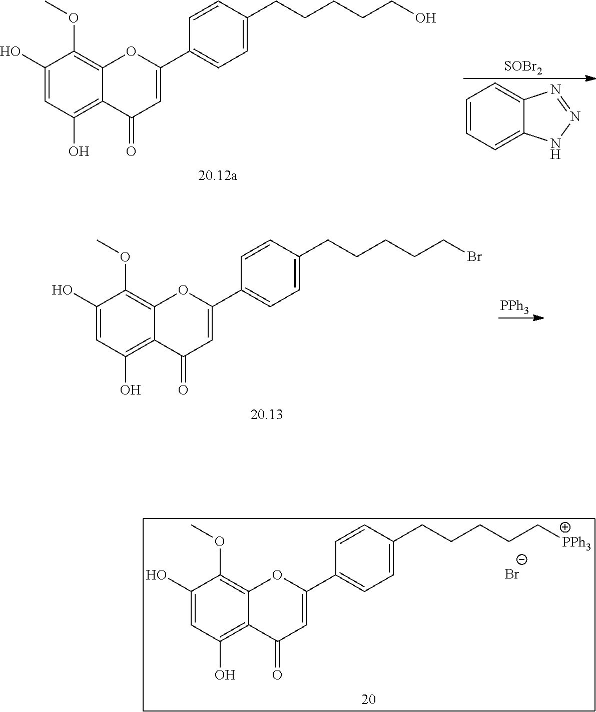

All solvents, reagents and compounds were purchased and used without further purification unless stated otherwise.

### Abbreviations

LiHMDS—Lithium bis(trimethylsilyl)amide
THF—Tetrahydrofuran
THP—Tetrahydropyran
Pd/C—Palladium on carbon (10 wt. % loading)
AcOH—Acetic acid
DCM—Dichloromethane
MeOH—Methanol
EtOH—Ethanol
Et₂NH—Diethylamine
TsOH—Toluenesulfonic acid

### 5,7-bis(benzyloxy)-8-methoxy-2-phenyl-4H-chromen-4-one (19.2)

(Bromomethyl)benzene (23.83 g, 16.57 mL, 5 Eq, 139.3 mmol) and K₂CO₃ (19.25 g, 5 Eq, 139.3 mmol) were added to a stirring solution of wogonin (7.920 g, 1 Eq, 27.86 mmol) in DMF (55 mL). The resulting suspension was stirred at 70° C. for 2 days. After this, additional benzyl bromide was added in the morning (4.765 g, 3.314 mL, 1 Eq, 27.86 mmol), and then again in the afternoon (2.383 g, 1.657 mL, 0.5 Eq, 13.93 mmol). After 4 days, the reaction still was not complete. Additional benzyl bromide (9.531 g, 6.628 mL, 2 Eq, 55.72 mmol), and K₂CO₃ (3.850 g, 1 Eq, 27.86 mmol) were added in the morning. Further benzyl bromide (2.383 g, 1.657 mL, 0.5 Eq, 13.93 mmol) was added in the afternoon. The following day the reaction was nearly complete. Additional benzyl bromide (1.191 g, 828.5 μL, 0.25 Eq, 6.965 mmol) was added in the morning, and the reaction had reached full completion by the afternoon.

The reaction was allowed to cool and was acidified to pH 1 with 1 M HCl. Then the aqueous layer was extracted with DCM (3×250 mL). The combined organic layers were dried with sodium sulphate. The solvent was removed in vacuo to give a crude brown solid (45.159 g). The product was dissolved in ˜120 mL DCM. The sample was split in half for 2×220 g silica columns, using EtOAc/Heptane as a eluent. Tubes of similar purity were combined from each column. This yielded two fractions of differing purity.

Fraction 1: 10.439 g, 97% pure

Fraction 2: 1.008 g, 92% pure.

Fraction 2 was recrystallized by dissolving in a little refluxing EtOAc as possible. As it cools down, more and more Heptane was added—inducing recrystallisation. The crystals were filtered and washed with additional heptane.

Crystals: 0.667 g, 100% pure.

The crystals and Fraction 1 were combined yielding a white solid (11.106 g, 23.909 mmol, 86% yield)

### 1-(4,6-bis(benzyloxy)-2-hydroxy-3-methoxyphenyl)ethan-1-one (20.3)

First pyridine (17.0 g, 17.4 mL, 20 Eq, 215 mmol) was added to 19.2 (5.00 g, 1 Eq, 10.8 mmol), and then quickly after a mixture of 50% aqueous sodium hydroxide solution (30.2 g 35 Eq, 4.9 mmol) was added. The mixture was vigorously stirred and treated with diethylene glycol (22.8 g, 20.6 mL, 20 Eq, 215 mmol). The mixture was heated to 100° C. and stirred for 17 hours. The mixture was cooled to <20° C., and the pH was adjusted to 1 with a 12N aqueous hydrochloric acid solution. More 1N HCl was added (5 mL) The aqueous portion was extracted with DCM (3×100 mL). The combined organic phase was washed with brine (50 mL), aqueous saturated sodium bicarbonate (120 mL) and dried with sodium sulfate, and the solvent was removed under reduced pressure. This gave the crude product as an brown solid/paste (6.409 g). Recrystallisation was attempted—dissolved in minimal refluxing EtOAc, and then a few drops of heptane is added. Mixture allowed to slowly cool down overnight to allow recrystallisation. The mother liquor was decanted off the yellow crystals, and the crystals were washed with heptane.

### 2-(Pent-4-en-1-yloxy)tetrahydro-2H-pyran (20.8a)

p-Toluenesulfonic acid monohydrate (0.45 g, 0.020 Eq, 2.4 mmol) and 3,4-dihydro-2H-pyran (20.3 g, 22 mL, 2 Eq, 241 mmol) were added to a solution of pent-4-en-1-ol (10.4 g, 12.5 mL, 1 Eq, 121 mmol) in DCM (50 mL) at 0° C. After 2 hours the reaction mixture was quenched with sat. NaHCO₃. (50 mL). It was then diluted with DCM (50 mL) and the layers were separated. Organic layer was washed with water (50 mL) and brine (50 mL), before it was dried with sodium sulfate, filtered and evaporated to dryness. The crude product was obtained as a dark oil (31.3 g). The crude product was applied on the column neat. It was then purified by column chromatography over 330 g of silica using Heptane/EtOAc as eluents. This obtained a pure fraction of 20.8a (16.88 g, 99.15 mmol, 82%) as a faintly yellow transparent oil.

### 4-(5-((Tetrahydro-2H-pyran-2-yl)oxy)pentyl)benzaldehyde (20.9a)

9-BBN (7.59 g, 62.2 mL, 0.5 molar, 1.48 Eq, 31.1 mmol) in THF was added to 20.8a (5.29 g, 1.47 Eq, 31.1 mmol) at room temperature under nitrogen flow in a flame-dried three-neck flask. The mixture was stirred at room temperature under nitrogen for 24 hours. Then aq. NaOH (1.24 g, 10.3 mL, 3 molar, 1.47 Eq, 31.0 mmol) was added. After the reaction mixture was left to stir for 20 minutes, 4-bromobenzaldehyde (3.90 g, 1 Eq, 21.1 mmol) was added with degassed THF (30 mL). The mixture was degassed for a further 30 mins. Pd(PPh₃)₄ (2.00 g, 0.0821 Eq, 1.73 mmol) added as solid. The mixture was degassed for 45 mins. The reaction was heated at 60° C. for 2 hours, after which the reaction was complete. The reaction mixture was diluted with EtOAc (750 mL) and washed with sat. NaHCO₃ (400 mL). The aqueous layer was extracted with EtOAc (2×150 mL). The organic layers were combined, dried with sodium sulfate, filtered and evaporated to dryness to yield brown oil with precipitate (13.984 g). This was purified over 330 g of silica using Heptane/EtOAc as eluents. This obtained an impure fraction of 20.9a (6.88 g, >quant. yield)

### (E)-1-(4,6-bis(benzyloxy)-2-hydroxy-3-methoxyphenyl)-3-(4-(5-((tetrahydro-2H-pyran-2-yl)oxy)pentyl)phenyl)prop-2-en-1-one (20.10)

Sodium methanolate (7.619 g, 26.12 mL, 5.4 molar, 35 Eq, 141.0 mmol) in MeOH was added portion-wise under nitrogen flow to an ice/water cooled (10° C.) suspension of 20.3 (1.525 g, 1 Eq, 4.030 mmol) and 20.9a (1.78 g, 1.60 Eq, 6.44 mmol) in 1,4-Dioxane (30 mL). This took 15 minutes, and the solution turned yellow after sodium methoxide solution was added. The mixture was allowed slowly to warm to room temperature and it was stirred for 20 hours under nitrogen atmosphere. The resultant dark reaction mixture was neutralised with (10% aq) citric acid, under ice-bath, until pH was neutral. The orange suspension was extracted with EtOAc (4×90 mL). Organic layers were combined, dried with sodium sulfate, filtered and concentrated. This gave a crude orange oil (3.88 g). This was purified by column chromatography over a 120 g column, using EtOAc/Heptane as an eluent. This gave a pure fraction of 20.10 (2.609 g, 4.03 mmol, 100% yield, >99% purity).

### 5,7-bis(benzyloxy)-2-(4-(5-hydroxypentyl)phenyl)-8-methoxy-4H-chromen-4-one (20.11)

A solution of 20.10 (1.95 g, 1 Eq, 3.06 mmol) and diiodine (50 mg, 0.064 Eq, 0.20 mmol) in DMSO (48 mL) were stirred at 120° C. for 20 hours. The reaction mixture was allowed to cool to room temperature, before pouring it to 460 mL of 2% sodium thiosulfate solution. Brown suspension formed. This was filtered. The residue was dissolved and washed from the filter using THF. This was dried with sodium sulphate and filtered. The solvent was removed which yielded a brown solid (4.35 g). This was purified by column chromatography over a 220 g silica column, using EtOAc/DCM as an eluent. This gave a fraction of impure 20.11 (1.692 g), with significant DMSO impurities. A second column over 80 g silica with THF/DCM eluent was carried out, this obtained a main fraction of 20.11 (0.928 g, 1.69 mmol, 55% yield, 100% purity). There is only 0.13 eq of DMSO by NMR.

### 5,7-dihydroxy-2-(4-(5-hydroxypentyl)phenyl)-8-methoxy-4H-chromen-4-one (20.12a)

Pd/C (1.31 g, 10% Wt, 0.731 Eq, 1.23 mmol) was added to a flask. A solution of 20.11 (0.9276 g, 1.0 Eq, 1.685 mmol) in THF (80 mL) was added. After purging with nitrogen, a hydrogen balloon was added. The reaction was monitored for 6 hours until completion by LCMS. After that hydrogen was removed and reaction mixture filtered over celite by THF (300 mL). This obtained a yellow/green solid (616 mg, 1.663 mmol, 99% yield, 90% purity). Major impurity is BHT from the THF.

### 2-(4-(5-bromopentyl)phenyl)-5,7-dihydroxy-8-methoxy-4H-chromen-4-one (20.13)

20.12a (616 mg, 1.0 Eq, 1.66 mmol) N,N-dimethylformamide (1.9 g, 2.0 mL, 16 Eq, 26 mmol), and DCM (10 mL) were transferred to a vial. The suspension was cooled to 0° C. and 1H-benzo[d][1,2,3]triazole (337 mg, 1.7 Eq, 2.83 mmol) and were added, followed by drop-wise addition of sulfurous dibromide (373 mg, 139 μL, 1.08 Eq, 1.80 mmol). The reaction was carefully monitored by LCMS to allow the reaction to progress to the optimum point—maximum amount of conversion to 20.13, and minimal conversion to the over-brominated byproducts. After 2 hours the reaction was quenched by with 16 mL of 1:1 water:sat. NaHCO₃. Additional LiCl (20%, 5 mL) was added. The aqueous layer was extracted with DCM (4×15 mL). The combined organic layers dried with sodium sulfate, filtered and concentrated to yield crude 20.13 as a brown oil (1.293 g). This was purified by column chromatography over a 40 g silica column, using EtOAc/DCM as an eluent. This yielded 20.13 as a yellow solid (240 mg, 553 μmol, 33% yield, 96% purity).

### (5-(4-(5,7-dihydroxy-8-methoxy-4-oxo-4H-chromen-2-yl)phenyl)pentyl)triphenylphosphonium bromide (20)

20.13 (113.5 mg, 1 Eq, 261.9 μmol) was dissolved in dry 1,4-dioxane (4 mL) in a microwave vial, by heating. Then triphenylphosphine (412.2 mg, 6 Eq, 1.572 mmol) and sodium iodide (3.926 mg, 0.1 Eq, 26.19 μmol) were added, the reaction purged with N₂. The additional reagents were dissolved by sonication. The vial was then heated at 100° C. for 3 days. The reaction was then cooled and precipitated with toluene (6 mL). It was filtered, washed with toluene (3×5 mL) and Et₂O (3×4 mL)

The solid was dissolved in methanol, and the solvent removed under reduced pressure. Precipitation was first tried with Et₂O. This gave 20 as a yellow powder (171.8 mg, 247 μmol, 94% yield, 95.8% purity). Compound 20 is SND 350.

### Examples—Biological Studies

Experimental Methodology

Antitumor activity of the compounds and doxorubicin as a positive control was assessed by using the CellTiter-Glow® Luminescent Cell Viability assay (Promega #G7572) according to the manufacturer's instructions. The compounds were tested at 5 or 6 concentrations in half-log increments (highest concentration 30 μM or 100 μM) in duplicate or triplicate well conditions.

Tumor cells were grown at 37° C. in a humidified atmosphere with 5% CO₂ in RPMI 1640 or DMEM medium, supplemented with 10% (v/v) fetal calf serum and 50 μg/ml gentamicin for up to 20 passages, and were passaged once or twice weekly. Cells were harvested using TrypLE or PBS buffer containing 1 mM EDTA, and the percentage of viable cells was determined using a CASY Model TI cell counter (OMNI Life Science). Cells were harvested from exponential phase cultures, counted and plated in 96 well flat-bottom microtiter plates at a cell density depending on the cell line's growth rate (4,000-20,000 cells/well depending on the cell line's growth rate, up to 60,000 for hematological cancer cell lines) in RPMI 1640 or DMEM medium supplemented with 10% (v/v) fetal calf serum and 50 μg/ml gentamicin (140 μl/well). Cultures were incubated at 37° C. and 5% CO₂ in a humidified atmosphere. After 24 h the test compounds were serially diluted in DMSO, transferred in cell culture medium, and 10 μl of test compounds or control medium were added to the assay plates and left on the cells for another 72 h. The DMSO concentration was kept constant at <0.3% v/v across the assay plate. Viability of cells was quantified by or CellTiter-Glow® Luminescent Cell Viability assay (Promega #G7572). Luminescence was measured with a microplate luminometer (Promega or PerkinElmer.

Sigmoidal concentration-response curves were fitted to the data points (test-versus-control, T/C values) obtained for each tumor model using GraphPad prism 5.02 software. IC₅₀ values are reported as absolute IC₅₀ values, being the concentration of test compound at the intersection of the concentration-response curves with T/C=50% Cell lines tested are presented in Table 1.

TABLE 1
Tumour cell lines type and designation

| Tumour model | Cell line |
|---|---|
| Brain | U-87 |
| Breast | MDA-MB-468 |
| Colon | KM12 |
| Lymphoma | U937 |
| | Farage |
| Melanoma | A375 |
| | A2058 |
| | SM-Mel-5 |

In Vivo Tolerability

To assess the toxicity of the test compounds in vivo and to determine the maximum tolerated dose 4 BALB/c Nude male mice, 4-9 weeks old were injected intraperitoneally (i.p) with various doses of the drug or vehicle 2.5% DMSO/5% EtOH/20% PEG200 in saline. The volume of the injection was 5 ml/kg. The administered doses were confirmed by LC-UV or LC-MS/MS.

Mice were monitored for body weight before each injection and twice a day for clinical signs of toxicity.

All procedures related to animal handling, care and the treatment in the study were performed according to the guidelines of the Association for Assessment and Accreditation of Laboratory Animal Care (AAALAC).

### Example 1. Activity Against Various Carcinoma Cell Lines

SND350 inhibited breast and colon carcinomas, lymphoma and melanoma cancer cell growth with IC50s below 10 μM, as presented in Table 2.

TABLE 2
IC50 values against various cancer cell lines

| Cell line/ | | | | | | | |
|---|---|---|---|---|---|---|---|
| IC50 (mM) | MDA-MB-468 | KM12 | U937 | Farage | A375 | SK-Mel-5 | A2058 |
| SND350 | 2.8 | 2.8 | 2.9 | 0.8 | 8.9 | 2.1 | 6.5 |

### Example 2. In Vivo Tolerability

In order to assess the in vivo toxicity of SND350 various doses of the drug were administered either as a single dose or repeat doses into nude mice. As a single i. p. administration the maximum tolerated dose was 40 mg/kg, whereas multiple administrations qod for a total of 7 injections were well tolerated at 10 mg/kg/inj.

It will be understood that the present invention has been described above byway of example only. The examples are not intended to limit the scope of the invention. Various modifications and embodiments can be made without departing from the scope and spirit of the invention, which is defined by the following claims only.

1. A compound of formula (I): 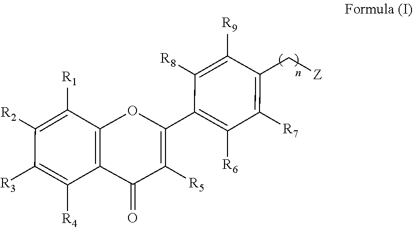
wherein:
Z is —[P(R¹¹)₃]X, wherein X is a counter anion;
R¹, R², and R⁴, independently, are selected from —OH, —O—C₁₋₄ alkyl, —OC(O)R₁₃, —OC(O)NHR¹³, and —OC(O)N(R¹³)₂; or
R¹ and R² together form —O—(C₁₋₃ alkylene)-O—; and R⁴ is selected from —OH, —O—C₁₋₄ alkyl, —OC(O)R₁₃, —OC(O)NHR¹³, and —OC(O)N(R¹³)₂;
R³, R⁵, R⁶, R⁷, R⁸, and R⁹, independently, are selected from H; halo; —CN; —NO₂; —R^{β}; —OH, —OR^{β}; —SH; —SR^{β}; —SOR^{β}; —SO₂H; —SO₂R^{β}; —SO₂NH₂; —SO₂NHR^{β}; —SO₂N(R^{β})₂; —NH₂; —NHR^{β}; —N(R^{β})₂; —CHO; —COR^{β}; —COOH; —COOR^{β}; —OCOR^{β}; and benzyl optionally substituted with 1-3 —R^{β};
each —R^{β} is independently selected from a C₁-C₆ alkyl, C₂-C₆ alkenyl, C₂-C₆ alkynyl or C₃-C₁₄ cyclic group, and wherein any —R^{β} may optionally be substituted with one or more C₁-C₄ alkyl, C₁-C₄ haloalkyl, C₃-C₇ cycloalkyl, —O(C₁-C₄ alkyl), —O(C₁-C₄ haloalkyl), —O(C₃-C₇ cycloalkyl), halo, —OH, —NH₂, —CN, —NO₂, —C≡CH, —CHO, —CON(CH₃)₂ or oxo (═O) groups;
each —R¹¹ is independently selected from H, C₁-C₆ alkyl, C₂-C₆ alkenyl, C₃-C₁₄ aryl group, or C₃-C₁₄ aliphatic cyclic group, and wherein any —R¹¹ may optionally be substituted with one or more C₁-C₄ alkyl, C₁-C₄ haloalkyl, C₃-C₇ cycloalkyl, —O(C₁-C₄ alkyl), —O(C₁-C₄ haloalkyl), —O(C₃-C₇ cycloalkyl), halo, —OH, —NH₂, —CN, —C≡CH or oxo (═O) groups
each —R¹³ is independently selected from a H, C₁-C₆ alkyl, C₂-C₆ alkenyl, C₂-C₆ alkynyl, C₃₋₁₄ cyclic group, halo, —NO₂, —CN, —OH, —NH₂, mercapto, formyl, carboxy, carbamoyl, C₁₋₆ alkoxy, C₁₋₆ alkylthio, —NH(C₁₋₆ alkyl), —N(C₁₋₆ alkyl)₂, C₁₋₆ alkylsulfinyl, C₁₋₆ alkylsulfonyl, or arylsulfonyl, wherein any —R¹³ may optionally be substituted with one or more —R¹⁴;
each R¹⁴ is independently selected from a C₁-C₆ alkyl, C₂-C₆ alkenyl, C₂-C₆ alkynyl, C₃₋₁₄ cyclic group, halo, —NO₂, —CN, —OH, —NH₂, mercapto, formyl, carboxy, carbamoyl, C₁₋₆ alkoxy, C₁₋₆ alkylthio, —NH(C₁₋₆ alkyl), —N(C₁₋₆ alkyl)₂, C₁₋₆ alkylsulfinyl, C₁₋₆ alkylsulfonyl, or arylsulfonyl, wherein any —R₁₄ may optionally be substituted with one or more —R₁₅;
each —R¹⁵ is independently selected from halogen, nitro, cyano, hydroxy, trifluoromethoxy, trifluoromethyl, amino, formyl, carboxy, carbamoyl, mercapto, sulfamoyl, methyl, ethyl, methoxy, ethoxy, acetyl, acetoxy, methylamino, ethylamino, dimethylamino, diethylamino, N-methyl-N-ethylamino, acetylamino, N-methylcarbamoyl N-ethylcarbamoyl N,N-dimethylcarbamoyl, N,N-diethylcarbamoyl, N-methyl-N-ethylcarbamoyl, methylthio, ethylthio, methylsulfinyl, ethylsulfinyl, mesyl ethylsulfonyl, methoxycarbonyl, ethoxycarbonyl, N-methylsulfamoyl N-ethylsulfamoyl N,N-dimethylsulfamoyl N,N-diethylsulfamoyl, N-methyl-N-ethylsulfamoyl, carbocyclyl, aryl, or heterocyclyl; and
n=1-10.

2. A compound as claimed in claim 1, wherein the compound is a compound of Formula 1A: 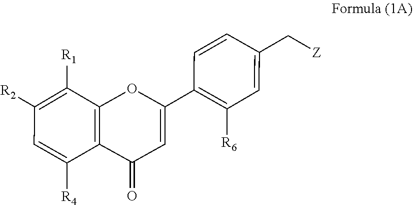
wherein:
Z is —[P(R¹¹)₃]X, wherein X is a counter anion;
R¹, R², and R⁴, independently, are selected from —OH, —O—C₁₋₄ alkyl, —OC(O)R₁₃, —OC(O)NHR¹³, —OC(O)N(R¹³)₂; or
R¹ and R² together form —O—(C₁₋₃ alkylene)-O—; and R⁴ is selected from —OH, —O—C₁₋₄ alkyl, —OC(O)R₁₃, —OC(O)NHR¹³, and —OC(O)N(R¹³)₂;
R⁶ is selected from H; halo; —CN; —NO₂; —R^{β}; —OH, —OR^{β}; —SH; —SR^{β}; —SOR^{β}; —SO₂H; —SO₂R^{β}; —SO₂NH₂; —SO₂NHR^{β}; —SO₂N(R^{β})₂; —NH₂; —NHR^{β}; —N(R^{β})₂; —CHO; —COR^{β}; —COOH; —COOR^{β}; —OCOR^{β}; and benzyl optionally substituted with 1-3 —R^{β};
each —R^{β} is independently selected from a C₁-C₆ alkyl, C₂-C₆ alkenyl, C₂-C₆ alkynyl or C₃-C₁₄ cyclic group, and wherein any —R^{β} may optionally be substituted with one or more C₁-C₄ alkyl, C₁-C₄ haloalkyl, C₃-C₇ cycloalkyl, —O(C₁-C₄ alkyl), —O(C₁-C₄ haloalkyl), —O(C₃-C₇ cycloalkyl), halo, —OH, —NH₂, —CN, —NO₂, —C≡CH, —CHO, —CON(CH₃)₂ or oxo (═O) groups;
each —R¹¹ is independently selected from H, C₁-C₆ alkyl, C₂-C₆ alkenyl, C₃-C₁₄ aryl group, or C₃-C₁₄ aliphatic cyclic group, and wherein any —R¹¹ may optionally be substituted with one or more C₁-C₄ alkyl, C₁-C₄ haloalkyl, C₃-C₇ cycloalkyl, —O(C₁-C₄ alkyl), —O(C₁-C₄ haloalkyl), —O(C₃-C₇ cycloalkyl), halo, —OH, —NH₂, —CN, —C≡CH or oxo (═O) groups
each —R¹³ is independently selected from a H, C₁-C₆ alkyl, C₂-C₆ alkenyl, C₂-C₆ alkynyl, C₃₋₁₄ cyclic group, halo, —NO₂, —CN, —OH, —NH₂, mercapto, formyl, carboxy, carbamoyl, C₁₋₆ alkoxy, C₁₋₆ alkylthio, —NH(C₁₋₆ alkyl), —N(C₁₋₆ alkyl)₂, C₁₋₆ alkylsulfinyl, C₁₋₆ alkylsulfonyl, or arylsulfonyl, wherein any —R¹³ may optionally be substituted with one or more —R¹⁴;
each R¹⁴ is independently selected from a C₁-C₆ alkyl, C₂-C₆ alkenyl, C₂-C₆ alkynyl, C₃₋₁₄ cyclic group, halo, —NO₂, —CN, —OH, —NH₂, mercapto, formyl, carboxy, carbamoyl, C₁₋₆ alkoxy, C₁₋₆ alkylthio, —NH(C₁₋₆ alkyl), —N(C₁₋₆ alkyl)₂, C₁₋₆ alkylsulfinyl, C₁₋₆ alkylsulfonyl, or arylsulfonyl, wherein any —R₁₄ may optionally be substituted with one or more —R₁₅;
each —R¹⁵ is independently selected from halogen, nitro, cyano, hydroxy, trifluoromethoxy, trifluoromethyl, amino, formyl, carboxy, carbamoyl, mercapto, sulfamoyl, methyl, ethyl, methoxy, ethoxy, acetyl, acetoxy, methylamino, ethylamino, dimethylamino, diethylamino, N-methyl-N-ethylamino, acetylamino, N-methylcarbamoyl N-ethylcarbamoyl N,N-dimethylcarbamoyl, N,N-diethylcarbamoyl, N-methyl-N-ethylcarbamoyl, methylthio, ethylthio, methylsulfinyl, ethylsulfinyl, mesyl ethylsulfonyl, methoxycarbonyl, ethoxycarbonyl, N-methylsulfamoyl N-ethylsulfamoyl N,N-dimethylsulfamoyl N,N-diethylsulfamoyl, N-methyl-N-ethylsulfamoyl, carbocyclyl, aryl, or heterocyclyl; and
n=1-10.

3. A compound as claimed in claim 1 or claim 2, wherein Z is —[P(R¹¹)₃]X, wherein each —R¹¹ is independently a C₃-C₁₄ aryl group; and wherein any —R¹¹ may optionally be substituted with one or more C₁-C₄ alkyl, halo, —OH, —NH₂, —CN, —C≡CH or oxo (═O) groups.

4. A compound as claimed in any one or more of the preceding claims, wherein each R¹¹ is phenyl.

5. A compound as claimed in any one or more of the preceding claims, wherein n is 3-6, or n is 4 or 5.

6. A compound as claimed in any one or more of the preceding claims, wherein R³, R⁵, R⁶, R⁷, R⁸, and R⁹, independently, are selected from H; halo; —CN; —NO₂; —R^{β}; —SH; —SR^{β}; —SOR^{β}; —SO₂H; —SO₂R^{β}; —SO₂NH₂; —SO₂NHR^{β}; —SO₂N(R^{β})₂; —NH₂; —NHR^{β}; —N(R^{β})₂; —CHO; —COR^{β}; —COOH; and —COOR^{β}; and benzyl optionally substituted with 1-3 —R^{β}.

7. A compound as claimed in any one or more of the preceding claims, wherein R³, R⁵, R⁶, R⁷, R⁸, and R⁹, independently, are selected from H; halo; —CN; —NO₂; —R^{β}; —NH₂; —NHR^{β}; —N(R^{β})₂; —CHO; —COR^{β}; —COOH; —COOR^{β}; and —OCOR^{β}.

8. A compound as claimed in any one or more of the preceding claims, wherein R³, R⁵, R⁶, R⁷, R⁸, and R⁹, independently, are selected from H; halo; —CN; —NO₂; —R^{β}; —NH₂; —NHR^{β}; —N(R^{β})₂; and —CHO.

9. A compound as claimed in any one or more of claims 1 to 6, wherein R³, R⁵, R⁶, R⁷, R⁸, and R⁹, independently, are selected from H; halo; —CN; —NO₂; —SH; —SO₂H; and —NH₂.

10. A compound as claimed in any one or more of the preceding claims, wherein R¹, R², and R⁴, independently, are selected from —OH, —O—C₁₋₄ alkyl, and —OC(O)R₁₃.

11. A compound as claimed in any of the preceding claims, wherein R¹, R², and R⁴, independently, are selected from —OH, and —O—C₁₋₄ alkyl.

12. A compound as claimed in any of claims 1 to 5, wherein R¹, R², and R⁴, independently, are selected from —OH, —O—C₁₋₄ alkyl, —OC(O)R₁₃, —OC(O)NHR¹³, —OC(O)N(R¹³)₂; and R³, R⁵, R⁶, R⁷, R⁸, and R⁹, independently, are selected from H; halo; —CN; —NO₂; —SH; —SO₂H; and —NH₂.

13. A compound as claimed in claim 1 selected from the following:

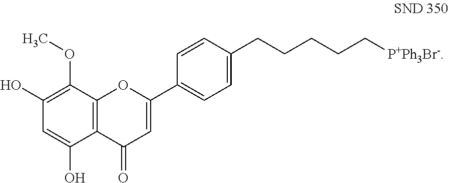

14. A pharmaceutically acceptable multi-salt, solvate or prodrug of a compound as defined in any one of claims 1 to 13.

15. A pharmaceutical composition comprising a compound as defined in any one of claims 1 to 13, or a pharmaceutically acceptable multi-salt, solvate or prodrug as defined in claim 14, and a pharmaceutically acceptable excipient.

16. A compound as defined in any one of claims 1 to 13, or a pharmaceutically acceptable multi-salt, solvate or prodrug as defined in claim 14, or a pharmaceutical composition as defined in claim 15, for use in medicine.

17. A compound as defined in any one of claims 1 to 13, or a pharmaceutically acceptable multi-salt, solvate or prodrug as defined in claim 14, or a pharmaceutical composition as defined in claim 15, for use treating or preventing cancer.

18. A method of treatment or prevention of a disease, disorder or condition, the method comprising the step of administering an effective amount of a compound as defined in any one of claims 1 to 13, or a pharmaceutically acceptable multi-salt, solvate or prodrug as defined in claim 14 or a pharmaceutical composition as defined in claim 15, to thereby treat or prevent the disease, disorder or condition.

19. A method of treatment as claimed in claim 18, wherein the disease, disorder or condition is cancer.

20. A compound, a pharmaceutically acceptable multi-salt, solvate or prodrug, or a pharmaceutical composition for use according to claim 17, or a method of treatment according to claim 19, wherein the cancer is brain cancer, breast cancer, colon cancer, leukaemia, lymphoma, or melanoma.
